(19) United States
(12) Patent Application Publication
(10) Pub. No.: US 2022/0305021 A1
(43) Pub. Date: Sep. 29, 2022

(54) METHODS OF TREATING AND/OR PREVENTING PSORIASIS

(71) Applicant: Athenex, Inc., Buffalo (US)

(72) Inventors: Johnson Yiu-Nam LAU, Houston, TX (US); Wing Kai CHAN, Hong Kong (CN); Min-Fun Rudolf KWAN, Summit, NJ (US)

(21) Appl. No.: 17/616,238

(22) PCT Filed: Jun. 5, 2020
(86) PCT No.: PCT/US2020/036238

§ 371 Date: Dec. 3, 2021

Related U.S. Application Data
(60) Provisional application No. 62/857,796, filed on Jun. 5, 2019.

(51) Int. Cl. A61K 31/5377; A61P 17/06; A61K 9/06; A61K 9/00

(52) U.S. Cl. CPC A61K 31/5377; A61P 17/06; A61K 9/06; A61K 9/0014

(57) ABSTRACT

The disclosure pertains to methods of treating and/or preventing psoriasis, comprising administering a therapeutically effective amount of KX-01,

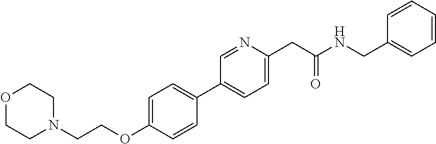 [SMILES: O=C(Cc1ccc(-c2ccc(OCCN3CCOCC3)cc2)cn1)NCc1ccccc1]

or a pharmaceutically acceptable salt thereof, to a subject in need thereof.

### RELATED APPLICATIONS

This application claims priority to, and the benefit of U.S. Provisional Application No. 62/857,796, filed on Jun. 5, 2019, the entire contents of which are incorporated herein in their entirety.

### BACKGROUND

Psoriasis is a chronic autoimmune skin disease that speeds up the growth cycle of skin cells. Psoriasis causes localized or generalized patches of red papules and plaques, covered with white or silver scales and itching, psoriasis may affect 100 million individuals worldwide.

Psoriasis is most common on the scalp, elbows, knees, and lower back. Psoriasis may present itself at any time, including early adulthood (e.g., 15 to 35 years old). The treatments for psoriasis include steroid creams, occlusion, light therapy, and oral medications. Psoriasis has been associated with other serious health conditions, including but not limited to diabetes, heart disease, and depression. There are five types of psoriasis: plaque psoriasis, guttate, inverse, pustular, and erythrodermic. Psoriasis affects approximately 1-3% of the general population worldwide, with chronic plaque psoriasis accounting for approximately 85-90% of all cases. Plaque-type psoriasis, or psoriasis vulgaris, is characterized mainly by the formation of inflamed, raised plaques that constantly shed scales derived from excessive growth of skin epithelial cells.

Traditional systemic therapies for psoriasis (e.g., methotrexate, cyclosporin A. retinoids or photochemotherapy with psoralens and ultraviolet A [PUVA]) have a potential for long-term toxicity and may not always provide sufficient improvement of the disease. There remains a need for new therapies that are effective and have less side effects for the treatment of psoriasis. The present disclosure addresses the need.

### SUMMARY

In one aspect, this disclosure pertains at least in part, to a method of treating and/or preventing psoriasis, comprising administering to a subject in need thereof a therapeutically effective amount of KX-01:

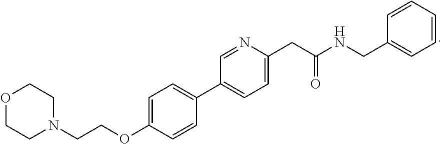 [SMILES: O=C(Cc1ccc(-c2ccc(OCCN3CCOCC3)cc2)cn1)NCc1ccccc1]

In one aspect, KX-01 is administered to an affected area of the subject at a dose from about 0.01 mg to about 10 mg.

In one aspect, KX-01 is administered to an affected area of the subject at a dose from about 0.1 mg to about 10 mg.

In one aspect, KX-01 is administered to an affected area of the subject at a dose from about 0.2 mg to about 5 mg.

In one aspect, KX-01 is administered to an affected area of the subject at a dose from about 0.5 mg to about 2.5 mg.

In one aspect, KX-01 is administered to an affected area of the subject at a dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.11 mg, about 0.12 mg, about 0.13 mg, about 0.14 mg, about 0.15 mg, about 0.16 mg, about 0.17 mg, about 0.18 mg, about 0.19 mg, about 0.20 mg, about 0.21 mg, about 0.22 mg, about 0.23 mg, about 0.24 mg, about 0.25 mg, about 0.26 mg, about 0.27 mg, about 0.28 mg, about 0.29 mg, about 0.3 mg, about 0.4 mg, or about 0.5 mg.

In one aspect, KX-01 is administered to an affected area of the subject at a dose of about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 4 mg, or about 5 mg.

In one aspect, KX-01 is administered to an affected area of the subject at a dose of about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, or about 2.5 mg.

In one aspect, KX-01 is administered to an affected area of the subject at a dose from about 0.1 mg/g to about 20 mg/g.

In one aspect, KX-01 is administered to an affected area of the subject at a dose from about 0.01 mg/g to about 10 mg/g.

In one aspect, KX-01 is administered to an affected area of the subject at a dose of about 0.1 mg/g.

In one aspect, KX-01 is administered to an affected area of the subject at a dose of about 1.0 mg/g.

In one aspect, KX-01 is administered to an affected area of the subject at a dose of about 10 mg/g.

In one aspect, KX-01 is administered to an affected area of the subject at a dose from about 0.0001 mg/cm² to about 5 mg/cm².

In one aspect, KX-01 is administered to an affected area of the subject at a dose from about 0.0003 mg/cm² to about 10 mg/cm².

In one aspect, KX-01 is administered to an affected area of the subject at a dose from about 0.001 mg/cm² to about 0.4 mg/cm².

In one aspect, KX-01 is administered to an affected area of the subject at a dose from about 0.005 mg/cm² to about 0.1 mg/cm² In one aspect, KX-01 is administered to an affected area of the subject at a dose from about 0.005 mg/cm² to about 0.02 mg/cm².

In one aspect, KX-01 is administered to an affected area of the subject at a dose from about 0.025 mg/cm² to about 0.1 mg/cm².

In one aspect, KX-01 is administered to an affected area of the subject at a dose of about 0.0001 mg/cm², about 0.0002 mg/cm², about 0.0003 mg/cm², about 0.0004 mg/cm², about 0.0005 mg/cm², about 0.0006 mg/cm², about 0.0007 mg/cm², about 0.0008 mg/cm², about 0.0009 mg/cm², about 0.001 mg/cm², about 0.002 mg/cm², about 0.003 mg/cm², about 0.004 mg/cm², about 0.005 mg/cm², about 0.006 mg/cm², about 0.007 mg/cm², about 0.008 mg/cm², about 0.009 mg/cm², about 0.01 mg/cm², about 0.015 mg/cm², about 0.02 mg/cm², about 0.025 mg/cm², about 0.03 mg/cm², about 0.035 mg/cm², or about 0.04 mg/cm².

In one aspect, KX-01 is administered to an affected area of the subject at a dose of about 0.001 mg/cm², about 0.002 mg/cm², about 0.003 mg/cm², about 0.004 mg/cm², about 0.005 mg/cm², about 0.006 mg/cm², about 0.007 mg/cm², about 0.008 mg/cm², about 0.009 mg/cm², about 0.01 mg/cm², about 0.02 mg/cm², about 0.03 mg/cm², about 0.04 mg/cm², about 0.05 mg/cm², about 0.06 mg/cm², about 0.07 mg/cm², about 0.08 mg/cm², about 0.09 mg/cm², about 0.1 mg/cm², about 0.15 mg/cm², about 0.2 mg/cm², about 0.25 mg/cm², about 0.3 mg/cm², about 0.35 mg/cm², or about 0.4 mg/cm².

In one aspect, KX-01 is administered to an affected area of the subject at a dose of about 0.005 mg/cm², about 0.006 mg/cm², about 0.007 mg/cm², about 0.008 mg/cm², about 0.009 mg/cm², about 0.01 mg/cm², about 0.015 mg/cm², about 0.02 mg/cm², about 0.025 mg/cm², about 0.03 mg/cm², about 0.035 mg/cm², about 0.04 mg/cm², about 0.045 mg/cm², about 0.05 mg/cm², about 0.055 mg/cm², about 0.06 mg/cm², about 0.065 mg/cm², about 0.07 mg/cm², about 0.075 mg/cm², about 0.08 mg/cm², about 0.085 mg/cm², about 0.09 mg/cm², about 0.095 mg/cm², or about 0.1 mg/cm².

In one aspect, the affected area of the subject is about 0.01 cm² to about 300 cm² In one aspect, the affected area of the subject is about 1 cm² to about 200 cm², about 1 cm² to about 100 cm², about 1 cm² to about 75 cm², about 1 cm² to about 50 cm², or about 1 cm² to about 25 cm².

In one aspect, the affected area of the subject is about 10 cm² to about 200 cm², about 10 cm² to about 100 cm², about 10 cm² to about 75 cm², about 10 cm² to about 50 cm², or about 10 cm² to about 25 cm².

In one aspect, the affected area of the subject is about 25 cm² to about 200 cm², about 25 cm² to about 100 cm², about 25 cm² to about 75 cm², or about 25 cm² to about 50 cm² In one aspect, the affected area of the subject is about 25 cm² to about 100 cm², about 25 cm² to about 90 cm², about 25 cm² to about 80 cm², or about 25 cm² to about 70 cm², about 25 cm² to about 60 cm², about 25 cm² to about 50 cm², about 25 cm² to about 40 cm², or about 25 cm² to about 30 cm².

In one aspect, the affected area of the subject is about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², or about 100 cm².

In one aspect, the affected area of the subject is the skin.

In one aspect, the affected area of the subject is located at one or more locations independently selected from the scalp, forehead, forearm, face, nose, ears, eye lids, lips, neck, arms, elbows, hands, trunk, legs, knees, and feet.

In one aspect, the subject has more than one affected area.

In one aspect, the affected area is contiguous.

In one aspect, the affected area is non-contiguous.

In one aspect, KX-01 is administered once a week, once every three days, once every two days, once a day, twice a day, three times a day, or four times a day.

In one aspect, KX-01 is administered once a day or twice a day.

In one aspect, KX-01 is administered once a day.

In one aspect, KX-01 is administered for 1, 2, 3, 4, 5, 6, 7, 8, 9, 10, 11, 12, 13, 14, 15, 16, 17, 18, 19, 20, or 21 days.

In one aspect, KX-01 is administered for 1, 2, 3, 4, 5, 6, 7, 8, 9, 10, 11, 12, 13, or 14 days.

In one aspect, KX-01 is administered for 8, 9, 10, 11, 12, 13, or 14 days.

In one aspect, KX-01 is administered for 1, 2, 3, 4, 5, 6, or 7 days.

In one aspect, KX-01 is administered for 1, 2, 3, 4, or 5 days.

In one aspect, KX-01 is administered for 5 days.

In one aspect, KX-01 is administered for 5 consecutive days.

In one aspect, KX-01 is administered for 1, 2, 3, 4, 5, or 6 days per week.

In one aspect, KX-01 is administered for 2, 3, 4, 5, or 6 days per week.

In one aspect, KX-01 is administered for 1 week, 2 weeks, 3 weeks, 4 weeks or more, optionally followed by 1 week, 2 weeks, 3 weeks, 4 weeks or more during which period KX-01 is not administered, further optionally followed by administration of KX-01 for 1 week, 2 weeks, 3 weeks, 4 weeks or more.

In one aspect, KX-01 is administered for 2 weeks, optionally followed by 1 week during which period KX-01 is not administered, further optionally followed by administration of KX-01 for 1 week or 2 weeks.

In one aspect, KX-01 is administered for 2 weeks, followed by 1 week during which period KX-01 is not administered, further followed by administration of KX-01 for 2 weeks.

In one aspect, KX-01 is administered for 4 weeks.

In one aspect, KX-01 is administered once or twice daily continuously for more than one day per week, followed by discontinuation of the administration for the rest of the week.

In one aspect, KX-01 is administered once or twice daily every other day.

In one aspect, KX-01 is administered once or twice daily every three days, every four days, every five days, every six days, or every seven days.

In one aspect, KX-01 is administered once or twice daily for two days in a row every three days, every four days, every five days, every six days, or every seven days.

In one aspect, KX-01 is administered once or twice daily for three days in a row every four days, every five days, every six days, or every seven days.

In one aspect, KX-01 is administered once or twice daily for four days in a row every five days, every six days, or every seven days.

In one aspect, KX-01 is administered until the psoriasis is fully treated.

In one aspect, KX-01 is administered topically.

In one aspect, the administration of KX-01 reduces the number and/or severity of local skin reactions or other adverse side effects in the subject compared to other treatments of psoriasis.

In one aspect, the administration of KX-01 reduces the number of the subjects that have local skin reactions or other adverse side effects compared to other treatments of psoriasis.

In one aspect, the local skin reaction is selected from the group selected from vesiculation, postulation, erosion, ulceration, redness, swelling, flaking, scaling, hard lumps, dryness, pus, and blistering.

In one aspect, the other side effect is selected from the group consisting of application site pain, application site pruritus, application site irritation, application site swelling, application site burning sensation, application site infection, periorbital edema, nasopharyngitis, chills, sore throat, drooping eyes, puffy eyes, hypopigmentation, hyperpigmentation, and headache.

In one aspect, this disclosure pertains at least in part, to KX-01 for use (e.g., topical use) in the treatment and/or prevention of psoriasis. In some aspects, KX-01 is for use at the doses, dosing schedules, and/or one or more affected area in a subject in need thereof as described herein.

In one aspect, this disclosure pertains at least in part, to use (e.g., topical use) of KX-01 in the treatment and/or prevention of psoriasis. In some aspects, KX-01 is used at the doses, dosing schedules, and/or one or more affected area in a subject in need thereof as described herein.

In one aspect, this disclosure pertains at least in part, to use of KX-01 in the manufacture of a medicament for the treatment and/or prevention of psoriasis. In some aspects, KX-01 is used at the doses, dosing schedules, and/or one or more affected area in a subject in need thereof as described herein.

### DESCRIPTION OF THE FIGURES

FIG. 1A depicts the study design for Stage I and Stage II for treatment of plaque-type psoriasis with KX-01.

FIG. 1B. depicts the study design for Stage III for treatment of plaque-type psoriasis with KX-01.

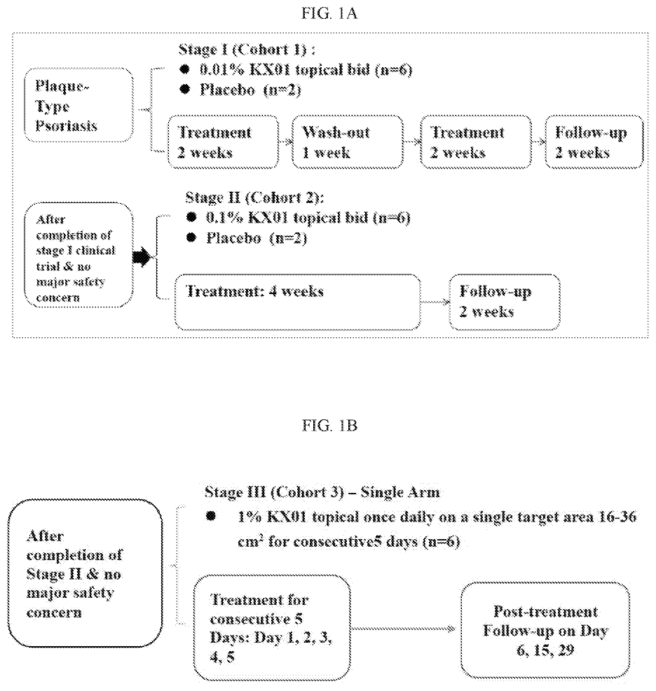

### DETAILED DESCRIPTION

The disclosure pertains to a method of treating and/or preventing psoriasis, comprising administering to a subject in need thereof a therapeutically effective amount of KX-01:

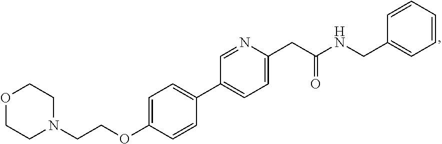 [SMILES: O=C(Cc1ccc(-c2ccc(OCCN3CCOCC3)cc2)cn1)NCc1ccccc1]

or a pharmaceutically acceptable salt thereof.

The disclosure pertains to a method of treating and/or preventing psoriasis, comprising administering to a subject in need thereof a therapeutically effective amount of KX-01:

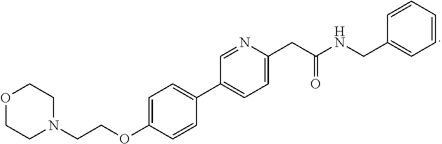 [SMILES: O=C(Cc1ccc(-c2ccc(OCCN3CCOCC3)cc2)cn1)NCc1ccccc1]

The disclosure pertains to a method of treating psoriasis comprising administering to a subject in need thereof a therapeutically effective amount of KX-01:

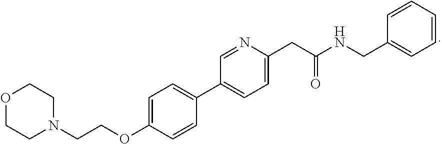 [SMILES: O=C(Cc1ccc(-c2ccc(OCCN3CCOCC3)cc2)cn1)NCc1ccccc1]

The disclosure pertains to a method of preventing psoriasis comprising administering to a subject in need thereof a therapeutically effective amount of KX-01:

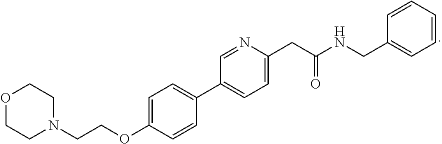 [SMILES: O=C(Cc1ccc(-c2ccc(OCCN3CCOCC3)cc2)cn1)NCc1ccccc1]

In some embodiments of the methods disclosed herein, KX-01 is administered to an affected area of the subject at a dose from about 0.1 mg to about 10 mg.

In some embodiments of the methods disclosed herein, KX-01 is administered to an affected area of the subject at a dose from about 0.2 mg to about 5 mg.

In some embodiments of the methods disclosed herein, KX-01 is administered to an affected area of the subject at a dose from about 0.5 mg to about 2.5 mg.

In some embodiments of the methods disclosed herein, KX-01 is administered to an affected area of the subject at a dose from about 0.01 mg to about 10 mg.

In some embodiments of the methods disclosed herein, KX-01 is administered to an affected area of the subject at a dose from about 0.025 mg to about 10 mg.

In some embodiments of the methods disclosed herein, KX-01 is administered to an affected area of the subject at a dose from about 0.25 mg to about 10 mg.

In some embodiments of the methods disclosed herein, KX-01 is administered to an affected area of the subject at a dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.11 mg, about 0.12 mg, about 0.13 mg, about 0.14 mg, about 0.15 mg, about 0.16 mg, about 0.17 mg, about 0.18 mg, about 0.19 mg, about 0.20 mg, about 0.21 mg, about 0.22 mg, about 0.23 mg, about 0.24 mg, about 0.25 mg, about 0.26 mg, about 0.27 mg, about 0.28 mg, about 0.29 mg, about 0.3 mg, about 0.4 mg, or about 0.5 mg.

In some embodiments of the methods disclosed herein, KX-01 is administered to an affected area of the subject at a dose from about 0.1 mg to about 9 mg, from about 0.1 mg to about 8 mg, from about 0.1 mg to about 7 mg, from about 0.1 mg to about 6 mg, from about 0.1 mg to about 5 mg, from about 0.1 mg to about 4 mg, from about 0.1 mg to about 3 mg, from about 0.1 mg to about 2 mg, from about 0.1 mg to about 1 mg, from about 0.1 mg to about 0.9 mg, from about 0.1 mg to about 0.8 mg, from about 0.1 mg to about 0.7 mg, from about 0.1 mg to about 0.6 mg, from about 0.1 mg to about 0.5 mg, from about 0.1 mg to about 0.4 mg, from about 0.1 mg to about 0.3 mg, or from about 0.1 mg to about 0.2 mg.

In some embodiments of the methods disclosed herein, KX-01 is administered to an affected area of the subject at a dose from about 0.2 mg to about 10 mg, from about 0.2 mg to about 9 mg, from about 0.2 mg to about 8 mg, from about 0.2 mg to about 7 mg, from about 0.2 mg to about 6 mg, from about 0.2 mg to about 5 mg, from about 0.2 mg to about 4 mg, from about 0.2 mg to about 3 mg, from about 0.2 mg to about 2 mg, from about 0.2 mg to about 1 mg, from about 0.2 mg to about 0.9 mg, from about 0.2 mg to about 0.8 mg, from about 0.2 mg to about 0.7 mg, from about 0.2 mg to about 0.6 mg, from about 0.2 mg to about 0.5 mg, from about 0.2 mg to about 0.4 mg, or from about 0.2 mg to about 0.3 mg,

In some embodiments of the methods disclosed herein, KX-01 is administered to an affected area of the subject at a dose from about 0.3 mg to about 10 mg, from about 0.3 mg to about 9 mg, from about 0.3 mg to about 8 mg, from about 0.3 mg to about 7 mg, from about 0.3 mg to about 6 mg, from about 0.3 mg to about 5 mg, from about 0.3 mg to about 4 mg, from about 0.3 mg to about 3 mg, from about 0.3 mg to about 2 mg, from about 0.3 mg to about 1 mg, from about 0.3 mg to about 0.9 mg, from about 0.3 mg to about 0.8 mg, from about 0.3 mg to about 0.7 mg, from about 0.3 mg to about 0.6 mg, from about 0.3 mg to about 0.5 mg, or from about 0.3 mg to about 0.4 mg.

In some embodiments of the methods disclosed herein, KX-01 is administered to an affected area of the subject at a dose from about 0.4 mg to about 10 mg, from about 0.4 mg to about 9 mg, from about 0.4 mg to about 8 mg, from about 0.4 mg to about 7 mg, from about 0.4 mg to about 6 mg, from about 0.4 mg to about 5 mg, from about 0.4 mg to about 4 mg, from about 0.4 mg to about 3 mg, from about 0.4 mg to about 2 mg, from about 0.4 mg to about 1 mg, from about 0.4 mg to about 0.9 mg, from about 0.4 mg to about 0.8 mg, from about 0.4 mg to about 0.7 mg, from about 0.4 mg to about 0.6 mg, or from about 0.4 mg to about 0.5 mg.

In some embodiments of the methods disclosed herein, KX-01 is administered to an affected area of the subject at a dose from about 0.5 mg to about 10 mg, from about 0.5 mg to about 9 mg, from about 0.5 mg to about 8 mg, from about 0.5 mg to about 7 mg, from about 0.5 mg to about 6 mg, from about 0.5 mg to about 5 mg, from about 0.5 mg to about 4 mg, from about 0.5 mg to about 3 mg, from about 0.5 mg to about 2 mg, from about 0.5 mg to about 1 mg, from about 0.5 mg to about 0.9 mg, from about 0.5 mg to about 0.8 mg, from about 0.5 mg to about 0.7 mg, or from about 0.5 mg to about 0.6 mg.

In some embodiments of the methods disclosed herein, KX-01 is administered to an affected area of the subject at a dose from about 0.6 mg to about 10 mg, from about 0.6 mg to about 9 mg, from about 0.6 mg to about 8 mg, from about 0.6 mg to about 7 mg, from about 0.6 mg to about 6 mg, from about 0.6 mg to about 5 mg, from about 0.6 mg to about 4 mg, from about 0.6 mg to about 3 mg, from about 0.6 mg to about 2 mg, from about 0.6 mg to about 1 mg, from about 0.6 mg to about 0.9 mg, from about 0.6 mg to about 0.8 mg, or from about 0.6 mg to about 0.7 mg.

In some embodiments of the methods disclosed herein, KX-01 is administered to an affected area of the subject at a dose from about 0.7 mg to about 10 mg, from about 0.7 mg to about 9 mg, from about 0.7 mg to about 8 mg, from about 0.7 mg to about 7 mg, from about 0.7 mg to about 6 mg, from about 0.7 mg to about 5 mg, from about 0.7 mg to about 4 mg, from about 0.7 mg to about 3 mg, from about 0.7 mg to about 2 mg, from about 0.7 mg to about 1 mg, from about 0.7 mg to about 0.9 mg, or from about 0.7 mg to about 0.8 mg.

In some embodiments of the methods disclosed herein, KX-01 is administered to an affected area of the subject at a dose from about 0.8 mg to about 10 mg, from about 0.8 mg to about 9 mg, from about 0.8 mg to about 8 mg, from about 0.8 mg to about 7 mg, from about 0.8 mg to about 6 mg, from about 0.8 mg to about 5 mg, from about 0.8 mg to about 4 mg, from about 0.8 mg to about 3 mg, from about 0.8 mg to about 2 mg, from about 0.8 mg to about 1 mg, or from about 0.8 mg to about 0.9 mg.

In some embodiments of the methods disclosed herein, KX-01 is administered to an affected area of the subject at a dose from about 0.9 mg to about 10 mg, from about 0.9 mg to about 9 mg, from about 0.9 mg to about 8 mg, from about 0.9 mg to about 7 mg, from about 0.9 mg to about 6 mg, from about 0.9 mg to about 5 mg, from about 0.9 mg to about 4 mg, from about 0.9 mg to about 3 mg, from about 0.9 mg to about 2 mg, or from about 0.9 mg to about 1 mg.

In some embodiments of the methods disclosed herein, KX-01 is administered to an affected area of the subject at a dose from about 1 mg to about 10 mg, from about 1 mg to about 9 mg, from about 1 mg to about 8 mg, from about 1 mg to about 7 mg, from about 1 mg to about 6 mg, from about 1 mg to about 5 mg, from about 1 mg to about 4 mg, from about 1 mg to about 3 mg, or from about 1 mg to about 2 mg.

In some embodiments of the methods disclosed herein, KX-01 is administered to an affected area of the subject at a dose from about 2 mg to about 10 mg, from about 2 mg to about 9 mg, from about 2 mg to about 8 mg, from about 2 mg to about 7 mg, from about 2 mg to about 6 mg, from about 2 mg to about 5 mg, from about 2 mg to about 4 mg, or from about 2 mg to about 3 mg.

In some embodiments of the methods disclosed herein, KX-01 is administered to an affected area of the subject at a dose from about 0.2 mg to about 10 mg, from about 0.3 mg to about 10 mg, from about 0.4 mg to about 10 mg, from about 0.5 mg to about 10 mg, from about 0.6 mg to about 10 mg, from about 0.7 mg to about 10 mg, from about 0.8 mg to about 10 mg, from about 0.9 mg to about 10 mg, from about 1 mg to about 10 mg, from about 2 mg to about 10 mg, from about 3 mg to about 10 mg, from about 4 mg to about 10 mg, from about 5 mg to about 10 mg, from about 6 mg to about 10 mg, from about 7 mg to about 10 mg, from about 8 mg to about 10 mg, or from about 9 mg to about 10 mg.

In some embodiments of the methods disclosed herein, KX-01 is administered to an affected area of the subject at a dose from about 0.1 mg to about 9 mg, from about 0.2 mg to about 9 mg, from about 0.3 mg to about 9 mg, from about 0.4 mg to about 9 mg, from about 0.5 mg to about 9 mg, from about 0.6 mg to about 9 mg, from about 0.7 mg to about 9 mg, from about 0.8 mg to about 9 mg, from about 0.9 mg to about 9 mg, from about 1 mg to about 9 mg, from about 2 mg to about 9 mg, from about 3 mg to about 9 mg, from about 4 mg to about 9 mg, from about 5 mg to about 9 mg, from about 6 mg to about 9 mg, from about 7 mg to about 9 mg, or from about 8 mg to about 9 mg.

In some embodiments of the methods disclosed herein, KX-01 is administered to an affected area of the subject at a dose from about 0.1 mg to about 8 mg, from about 0.2 mg to about 8 mg, from about 0.3 mg to about 8 mg, from about 0.4 mg to about 8 mg, from about 0.5 mg to about 8 mg, from about 0.6 mg to about 8 mg, from about 0.7 mg to about 8 mg, from about 0.8 mg to about 8 mg, from about 0.9 mg to about 8 mg, from about 1 mg to about 8 mg, from about 2 mg to about 8 mg, from about 3 mg to about 8 mg, from about 4 mg to about 8 mg, from about 5 mg to about 8 mg, from about 6 mg to about 8 mg, or from about 7 mg to about 8 mg.

In some embodiments of the methods disclosed herein, KX-01 is administered to an affected area of the subject at a dose from about 0.1 mg to about 7 mg, from about 0.2 mg to about 7 mg, from about 0.3 mg to about 7 mg, from about 0.4 mg to about 7 mg, from about 0.5 mg to about 7 mg, from about 0.6 mg to about 7 mg, from about 0.7 mg to about 7 mg, from about 0.8 mg to about 7 mg, from about 0.9 mg to about 7 mg, from about 1 mg to about 7 mg, from about 2 mg to about 7 mg, from about 3 mg to about 7 mg, from about 4 mg to about 7 mg, from about 5 mg to about 7 mg, or from about 6 mg to about 7 mg.

In some embodiments of the methods disclosed herein, KX-01 is administered to an affected area of the subject at a dose from about 0.1 mg to about 6 mg, from about 0.2 mg to about 6 mg, from about 0.3 mg to about 6 mg, from about 0.4 mg to about 6 mg, from about 0.5 mg to about 6 mg, from about 0.6 mg to about 6 mg, from about 0.7 mg to about 6 mg, from about 0.8 mg to about 6 mg, from about 0.9 mg to about 6 mg, from about 1 mg to about 6 mg, from about 2 mg to about 6 mg, from about 3 mg to about 6 mg, from about 4 mg to about 6 mg, or from about 5 mg to about 6 mg.

In some embodiments of the methods disclosed herein, KX-01 is administered to an affected area of the subject at a dose from about 0.1 mg to about 5 mg, from about 0.2 mg to about 5 mg, from about 0.3 mg to about 5 mg, from about 0.4 mg to about 5 mg, from about 0.5 mg to about 5 mg, from about 0.6 mg to about 5 mg, from about 0.7 mg to about 5 mg, from about 0.8 mg to about 5 mg, from about 0.9 mg to about 5 mg, from about 1 mg to about 5 mg, from about 2 mg to about 5 mg, from about 3 mg to about 5 mg, or from about 4 mg to about 5 mg.

In some embodiments of the methods disclosed herein, KX-01 is administered to an affected area of the subject at a dose from about 0.1 mg to about 4 mg, from about 0.2 mg to about 4 mg, from about 0.3 mg to about 4 mg, from about 0.4 mg to about 4 mg, from about 0.5 mg to about 4 mg, from about 0.6 mg to about 4 mg, from about 0.7 mg to about 4 mg, from about 0.8 mg to about 4 mg, from about 0.9 mg to about 4 mg, from about 1 mg to about 4 mg, from about 2 mg to about 4 mg, or from about 3 mg to about 4 mg.

In some embodiments of the methods disclosed herein, KX-01 is administered to an affected area of the subject at a dose from about 0.1 mg to about 3 mg, from about 0.2 mg to about 3 mg, from about 0.3 mg to about 3 mg, from about 0.4 mg to about 3 mg, from about 0.5 mg to about 3 mg, from about 0.6 mg to about 3 mg, from about 0.7 mg to about 3 mg, from about 0.8 mg to about 3 mg, from about 0.9 mg to about 3 mg, from about 1 mg to about 3 mg, or from about 2 mg to about 3 mg.

In some embodiments of the methods disclosed herein, KX-01 is administered to an affected area of the subject at a dose from about 0.1 mg to about 2 mg, from about 0.2 mg to about 2 mg, from about 0.3 mg to about 2 mg, from about 0.4 mg to about 2 mg, from about 0.5 mg to about 2 mg, from about 0.6 mg to about 2 mg, from about 0.7 mg to about 2 mg, from about 0.8 mg to about 2 mg, from about 0.9 mg to about 2 mg, or from about 1 mg to about 2 mg.

In some embodiments of the methods disclosed herein, KX-01 is administered to an affected area of the subject at a dose from about 0.1 mg to about 1 mg, from about 0.2 mg to about 1 mg, from about 0.3 mg to about 1 mg, from about 0.4 mg to about 1 mg, from about 0.5 mg to about 1 mg, from about 0.6 mg to about 1 mg, from about 0.7 mg to about 1 mg, from about 0.8 mg to about 1 mg, or from about 0.9 mg to about 1 mg.

In some embodiments of the methods disclosed herein, KX-01 is administered to an affected area of the subject at a dose from about 0.2 mg to about 3 mg, from about 0.2 mg to about 2.9 mg, from about 0.2 mg to about 2.8 mg, from about 0.2 mg to about 2.7 mg, from about 0.2 mg to about 2.6 mg, from about 0.2 mg to about 2.5 mg, from about 0.2 mg to about 2.4 mg, from about 0.2 mg to about 2.3 mg, from about 0.2 mg to about 2.2 mg, from about 0.2 mg to about 2.1 mg, or from about 0.2 mg to about 2.0 mg.

In some embodiments of the methods disclosed herein, KX-01 is administered to an affected area of the subject at a dose from about 0.3 mg to about 3 mg, from about 0.3 mg to about 2.9 mg, from about 0.3 mg to about 2.8 mg, from about 0.3 mg to about 2.7 mg, from about 0.3 mg to about 2.6 mg, from about 0.3 mg to about 2.5 mg, from about 0.3 mg to about 2.4 mg, from about 0.3 mg to about 2.3 mg, from about 0.3 mg to about 2.2 mg, from about 0.3 mg to about 2.1 mg, or from about 0.3 mg to about 2.0 mg.

In some embodiments of the methods disclosed herein, KX-01 is administered to an affected area of the subject at a dose from about 0.4 mg to about 3 mg, from about 0.4 mg to about 2.9 mg, from about 0.4 mg to about 2.8 mg, from about 0.4 mg to about 2.7 mg, from about 0.4 mg to about 2.6 mg, from about 0.4 mg to about 2.5 mg, from about 0.4 mg to about 2.4 mg, from about 0.4 mg to about 2.3 mg, from about 0.4 mg to about 2.2 mg, from about 0.4 mg to about 2.1 mg, or from about 0.4 mg to about 2.0 mg.

In some embodiments of the methods disclosed herein, KX-01 is administered to an affected area of the subject at a dose from about 0.5 mg to about 3 mg, from about 0.5 mg to about 2.9 mg, from about 0.5 mg to about 2.8 mg, from about 0.5 mg to about 2.7 mg, from about 0.5 mg to about 2.6 mg, from about 0.5 mg to about 2.5 mg, from about 0.5 mg to about 2.4 mg, from about 0.5 mg to about 2.3 mg, from about 0.5 mg to about 2.2 mg, from about 0.5 mg to about 2.1 mg, or from about 0.5 mg to about 2.0 mg.

In some embodiments of the methods disclosed herein, KX-01 is administered to an affected area of the subject at a dose from about 0.2 mg to about 3 mg, from about 0.3 mg to about 3 mg, from about 0.4 mg to about 3 mg, from about 0.5 mg to about 3 mg, from about 0.6 mg to about 3 mg, from about 0.7 mg to about 3 mg, from about 0.8 mg to about 3 mg, from about 0.9 mg to about 3 mg, or from about 1 mg to about 3 mg.

In some embodiments of the methods disclosed herein, KX-01 is administered to an affected area of the subject at a dose from about 0.2 mg to about 2.9 mg, from about 0.3 mg to about 2.9 mg, from about 0.4 mg to about 2.9 mg, from about 0.5 mg to about 2.9 mg, from about 0.6 mg to about 2.9 mg, from about 0.7 mg to about 2.9 mg, from about 0.8 mg to about 2.9 mg, from about 0.9 mg to about 2.9 mg, or from about 1 mg to about 2.9 mg.

In some embodiments of the methods disclosed herein, KX-01 is administered to an affected area of the subject at a dose from about 0.2 mg to about 2.8 mg, from about 0.3 mg to about 2.8 mg, from about 0.4 mg to about 2.8 mg, from about 0.5 mg to about 2.8 mg, from about 0.6 mg to about 2.8 mg, from about 0.7 mg to about 2.8 mg, from about 0.8 mg to about 2.8 mg, from about 0.9 mg to about 2.8 mg, or from about 1 mg to about 2.8 mg.

In some embodiments of the methods disclosed herein, KX-01 is administered to an affected area of the subject at a dose from about 0.2 mg to about 2.7 mg, from about 0.3 mg to about 2.7 mg, from about 0.4 mg to about 2.7 mg, from about 0.5 mg to about 2.7 mg, from about 0.6 mg to about 2.7 mg, from about 0.7 mg to about 2.7 mg, from about 0.8 mg to about 2.7 mg, from about 0.9 mg to about 2.7 mg, or from about 1 mg to about 2.7 mg.

In some embodiments of the methods disclosed herein, KX-01 is administered to an affected area of the subject at a dose from about 0.2 mg to about 2.6 mg, from about 0.3 mg to about 2.6 mg, from about 0.4 mg to about 2.6 mg, from about 0.5 mg to about 2.6 mg, from about 0.6 mg to about 2.6 mg, from about 0.7 mg to about 2.6 mg, from about 0.8 mg to about 2.6 mg, from about 0.9 mg to about 2.6 mg, or from about 1 mg to about 2.6 mg.

In some embodiments of the methods disclosed herein, KX-01 is administered to an affected area of the subject at a dose from about 0.2 mg to about 2.5 mg, from about 0.3 mg to about 2.5 mg, from about 0.4 mg to about 2.5 mg, from about 0.5 mg to about 2.5 mg, from about 0.6 mg to about 2.5 mg, from about 0.7 mg to about 2.5 mg, from about 0.8 mg to about 2.5 mg, from about 0.9 mg to about 2.5 mg, or from about 1 mg to about 2.5 mg.

In some embodiments of the methods disclosed herein, KX-01 is administered to an affected area of the subject at a dose from about 0.2 mg to about 2.4 mg, from about 0.3 mg to about 2.4 mg, from about 0.4 mg to about 2.4 mg, from about 0.5 mg to about 2.4 mg, from about 0.6 mg to about 2.4 mg, from about 0.7 mg to about 2.4 mg, from about 0.8 mg to about 2.4 mg, from about 0.9 mg to about 2.4 mg, or from about 1 mg to about 2.4 mg.

In some embodiments of the methods disclosed herein, KX-01 is administered to an affected area of the subject at a dose from about 0.2 mg to about 2.3 mg, from about 0.3 mg to about 2.3 mg, from about 0.4 mg to about 2.3 mg, from about 0.5 mg to about 2.3 mg, from about 0.6 mg to about 2.3 mg, from about 0.7 mg to about 2.3 mg, from about 0.8 mg to about 2.3 mg, from about 0.9 mg to about 2.3 mg, or from about 1 mg to about 2.3 mg.

In some embodiments of the methods disclosed herein, KX-01 is administered to an affected area of the subject at a dose from about 0.2 mg to about 2.2 mg, from about 0.3 mg to about 2.2 mg, from about 0.4 mg to about 2.2 mg, from about 0.5 mg to about 2.2 mg, from about 0.6 mg to about 2.2 mg, from about 0.7 mg to about 2.2 mg, from about 0.8 mg to about 2.2 mg, from about 0.9 mg to about 2.2 mg, or from about 1 mg to about 2.2 mg.

In some embodiments of the methods disclosed herein, KX-01 is administered to an affected area of the subject at a dose from about 0.2 mg to about 2.1 mg, from about 0.3 mg to about 2.1 mg, from about 0.4 mg to about 2.1 mg, from about 0.5 mg to about 2.1 mg, from about 0.6 mg to about 2.1 mg, from about 0.7 mg to about 2.1 mg, from about 0.8 mg to about 2.1 mg, from about 0.9 mg to about 2.1 mg, or from about 1 mg to about 2.1 mg.

In some embodiments of the methods disclosed herein, KX-01 is administered to an affected area of the subject at a dose from about 0.2 mg to about 2 mg, from about 0.3 mg to about 2 mg, from about 0.4 mg to about 2 mg, from about 0.5 mg to about 2 mg, from about 0.6 mg to about 2 mg, from about 0.7 mg to about 2 mg, from about 0.8 mg to about 2 mg, from about 0.9 mg to about 2 mg, or from about 1 mg to about 2 mg.

In some embodiments of the methods disclosed herein, KX-01 is administered to an affected area of the subject at a dose of about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 4 mg, or about 5 mg.

In some embodiments of the methods disclosed herein, KX-01 is administered to an affected area of the subject at a dose of about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, or about 2.5 mg.

In some embodiments of the methods disclosed herein, KX-01 is administered to an affected area of the subject at a dose of about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, or about 2.5 mg.

In some embodiments of the methods disclosed herein, KX-01 is administered to an affected area of the subject at a dose from about 0.1 mg/g to about 20 mg/g.

In some embodiments of the methods disclosed herein, KX-01 is administered to an affected area of the subject at a dose from about 0.5 mg/g to about 20 mg/g.

In some embodiments of the methods disclosed herein, KX-01 is administered to an affected area of the subject at a dose from about 1 mg/g to about 20 mg/g.

In some embodiments of the methods disclosed herein, KX-01 is administered to an affected area of the subject at a dose from about 1 mg/g to about 15 mg/g.

In some embodiments of the methods disclosed herein, KX-01 is administered to an affected area of the subject at a dose from about 5 mg/g to about 15 mg/g.

In some embodiments of the methods disclosed herein, KX-01 is administered to an affected area of the subject at a dose from about 7.5 mg/g to about 12.5 mg/g.

In some embodiments of the methods disclosed herein, KX-01 is administered to an affected area of the subject at a dose from about 8 mg/g to about 12 mg/g.

In some embodiments of the methods disclosed herein, KX-01 is administered to an affected area of the subject at a dose from about 0.01 mg/g to about 10 mg/g.

In some embodiments of the methods disclosed herein, KX-01 is administered to an affected area of the subject at a dose from about 0.05 mg/g to about 10 mg/g.

In some embodiments of the methods disclosed herein, KX-01 is administered to an affected area of the subject at a dose from about 0.05 mg/g to about 5 mg/g.

In some embodiments of the methods disclosed herein, KX-01 is administered to an affected area of the subject at a dose from about 0.05 mg/g to about 2.5 mg/g.

In some embodiments of the methods disclosed herein, KX-01 is administered to an affected area of the subject at a dose from about 0.05 mg/g to about 0.25 mg/g.

In some embodiments of the methods disclosed herein, KX-01 is administered to an affected area of the subject at a dose from about 0.1 mg/g to about 10 mg/g.

In some embodiments of the methods disclosed herein, KX-01 is administered to an affected area of the subject at a dose from about 0.2 mg/g to about 5 mg/g.

In some embodiments of the methods disclosed herein, KX-01 is administered to an affected area of the subject at a dose from about 0.5 mg/g to about 2.5 mg/g.

In some embodiments of the methods disclosed herein, KX-01 is administered to an affected area of the subject at a dose of about 0.1 mg/g.

In some embodiments of the methods disclosed herein, KX-01 is administered to an affected area of the subject at a dose of about 1.0 mg/g.

In some embodiments of the methods disclosed herein, KX-01 is administered to an affected area of the subject at a dose of about 10 mg/g.

In some embodiments of the methods disclosed herein, KX-01 is administered to an affected area of the subject at a dose of about 0.005 mg/g, about 0.01 mg/g, about 0.05 mg/g, about 0.1 mg/g, about 0.5 mg/g, about 1.0 mg/g, about 2.0 mg/g, about 3.0 mg/g, about 4.0 mg/g, about 5.0 mg/g, about 6.0 mg/g, about 7.0 mg/g, about 8.0 mg/g, about 9.0 mg/g, about 10.0 mg/g, about 11.0 mg/g, about 12.0 mg/g, about 13.0 mg/g, about 14.0 mg/g, about 15.0 mg/g, about 16.0 mg/g, about 17.0 mg/g, about 18.0 mg/g, about 19.0 mg/g, or about 20.0 mg/g.

In some embodiments of the methods disclosed herein, KX-01 is administered to an affected area of the subject at a dose from about 0.0001 mg/cm² to about 5 mg/cm².

In some embodiments of the methods disclosed herein, KX-01 is administered to an affected area of the subject at a dose from about 0.0001 mg/cm² to about 2.5 mg/cm².

In some embodiments of the methods disclosed herein, KX-01 is administered to an affected area of the subject at a dose from about 0.0001 mg/cm² to about 1 mg/cm².

In some embodiments of the methods disclosed herein, KX-01 is administered to an affected area of the subject at a dose from about 0.0001 mg/cm² to about 0.5 mg/cm².

In some embodiments of the methods disclosed herein, KX-01 is administered to an affected area of the subject at a dose from about 0.0001 mg/cm² to about 0.1 mg/cm².

In some embodiments of the methods disclosed herein, KX-01 is administered to an affected area of the subject at a dose from about 0.0001 mg/cm² to about 0.01 mg/cm².

In some embodiments of the methods disclosed herein, KX-01 is administered to an affected area of the subject at a dose from about 0.0002 mg/cm² to about 10 mg/cm².

In some embodiments of the methods disclosed herein, KX-01 is administered to an affected area of the subject at a dose from about 0.0002 mg/cm² to about 5 mg/cm².

In some embodiments of the methods disclosed herein, KX-01 is administered to an affected area of the subject at a dose from about 0.0002 mg/cm² to about 2.5 mg/cm².

In some embodiments of the methods disclosed herein, KX-01 is administered to an affected area of the subject at a dose from about 0.0002 mg/cm² to about 1 mg/cm².

In some embodiments of the methods disclosed herein, KX-01 is administered to an affected area of the subject at a dose from about 0.0002 mg/cm² to about 0.1 mg/cm².

In some embodiments of the methods disclosed herein, KX-01 is administered to an affected area of the subject at a dose from about 0.0002 mg/cm² to about 0.01 mg/cm².

In some embodiments of the methods disclosed herein, KX-01 is administered to an affected area of the subject at a dose from about 0.0003 mg/cm² to about 10 mg/cm².

In some embodiments of the methods disclosed herein, KX-01 is administered to an affected area of the subject at a dose from about 0.001 mg/cm² to about 0.4 mg/cm².

In some embodiments of the methods disclosed herein, KX-01 is administered to an affected area of the subject at a dose from about 0.005 mg/cm² to about 0.1 mg/cm².

In some embodiments of the methods disclosed herein, KX-01 is administered to an affected area of the subject at a dose from about 0.005 mg/cm² to about 0.02 mg/cm².

In some embodiments of the methods disclosed herein, KX-01 is administered to an affected area of the subject at a dose from about 0.025 mg/cm² to about 0.1 mg/cm².

In some embodiments of the methods disclosed herein, KX-01 is administered to an affected area of the subject at a dose of about 0.0001 mg/cm², about 0.0002 mg/cm², about 0.0003 mg/cm², about 0.0004 mg/cm², about 0.0005 mg/cm², about 0.0006 mg/cm², about 0.0007 mg/cm², about 0.0008 mg/cm², about 0.0009 mg/cm², about 0.001 mg/cm², about 0.002 mg/cm², about 0.003 mg/cm², about 0.004 mg/cm², about 0.005 mg/cm², about 0.006 mg/cm², about 0.007 mg/cm², about 0.008 mg/cm², about 0.009 mg/cm², about 0.01 mg/cm², about 0.015 mg/cm², about 0.02 mg/cm², about 0.025 mg/cm², about 0.03 mg/cm², about 0.035 mg/cm², or about 0.04 mg/cm².

In some embodiments of the methods disclosed herein, KX-01 is administered to an affected area of the subject at a dose of about 0.001 mg/cm², about 0.002 mg/cm², about 0.003 mg/cm², about 0.004 mg/cm², about 0.005 mg/cm², about 0.006 mg/cm², about 0.007 mg/cm², about 0.008 mg/cm², about 0.009 mg/cm², about 0.01 mg/cm², about 0.02 mg/cm², about 0.03 mg/cm², about 0.04 mg/cm², about 0.05 mg/cm², about 0.06 mg/cm², about 0.07 mg/cm², about 0.08 mg/cm², about 0.09 mg/cm², about 0.1 mg/cm², about 0.15 mg/cm², about 0.2 mg/cm², about 0.25 mg/cm², about 0.3 mg/cm², about 0.35 mg/cm², or about 0.4 mg/cm².

In some embodiments of the methods disclosed herein, KX-01 is administered to an affected area of the subject at a dose of about 0.005 mg/cm², about 0.006 mg/cm², about 0.007 mg/cm², about 0.008 mg/cm², about 0.009 mg/cm², about 0.01 mg/cm², about 0.015 mg/cm², about 0.02 mg/cm², about 0.025 mg/cm², about 0.03 mg/cm², about 0.035 mg/cm², about 0.04 mg/cm², about 0.045 mg/cm², about 0.05 mg/cm², about 0.055 mg/cm², about 0.06 mg/cm², about 0.065 mg/cm², about 0.07 mg/cm², about 0.075 mg/cm², about 0.08 mg/cm², about 0.085 mg/cm², about 0.09 mg/cm², about 0.095 mg/cm², or about 0.1 mg/cm².

In some embodiments of the methods disclosed herein, KX-01 is administered to an affected area of the subject at a dose of about 0.025 mg/cm², about 0.02 mg/cm², about 0.015 mg/cm², about 0.01 mg/cm², about 0.005 mg/cm², about 0.002 mg/cm², about 0.001 mg/cm², about 0.0005 mg/cm², about 0.0002 mg/cm², or about 0.0001 mg/cm².

In some embodiments of the methods disclosed herein, KX-01 is administered to an affected area of the subject at a dose of about 0.025 mg/cm².

In some embodiments of the methods disclosed herein, KX-01 is administered to an affected area of the subject at a dose of about 0.02 mg/cm².

In some embodiments of the methods disclosed herein, KX-01 is administered to an affected area of the subject at a dose of about 0.015 mg/cm².

In some embodiments of the methods disclosed herein, KX-01 is administered to an affected area of the subject at a dose of about 0.01 mg/cm².

In some embodiments of the methods disclosed herein, KX-01 is administered to an affected area of the subject at a dose of about 0.005 mg/cm².

In some embodiments of the methods disclosed herein, KX-01 is administered to an affected area of the subject at a dose of about 0.002 mg/cm².

In some embodiments of the methods disclosed herein, KX-01 is administered to an affected area of the subject at a dose of about 0.001 mg/cm².

In some embodiments of the methods disclosed herein, KX-01 is administered to an affected area of the subject at a dose of about 0.0005 mg/cm².

In some embodiments of the methods disclosed herein, KX-01 is administered to an affected area of the subject at a dose of about 0.0002 mg/cm².

In some embodiments of the methods disclosed herein, KX-01 is administered to an affected area of the subject at a dose of about 0.0001 mg/cm².

In some embodiments of the methods disclosed herein, KX-01 is administered to an affected area of the subject that is about 0.01 cm² to about 300 cm².

In some embodiments of the methods disclosed herein, KX-01 is administered to an affected area of the subject that is about 0.01 cm² to about 200 cm², about 0.01 cm² to about 100 cm², about 0.01 cm² to about 75 cm², about 0.01 cm² to about 50 cm², or about 0.01 cm² to about 25 cm².

In some embodiments of the methods disclosed herein, KX-01 is administered to an affected area of the subject that is about 0.1 cm² to about 625 cm², is about 0.1 cm² to about 300 cm 2, about 0.1 cm² to about 150 cm², about 0.1 cm² to about 100 cm², about 0.1 cm² to about 75 cm², or about 0.1 cm² to about 50 cm².

In some embodiments of the methods disclosed herein, KX-01 is administered to an affected area of the subject that is about 0.1 cm² to about 300 cm², is about 0.1 cm² to about 200 cm², about 0.1 cm² to about 100 cm², about 0.1 cm² to about 75 cm², about 0.1 cm² to about 50 cm 2, or about 0.1 cm² to about 25 cm².

In some embodiments of the methods disclosed herein, KX-01 is administered to an affected area of the subject that is about 10 cm² to about 625 cm², about 10 cm² to about 300 cm², is about 10 cm² to about 200 cm², about 10 cm² to about 100 cm², about 10 cm² to about 75 cm², about 10 cm² to about 50 cm², or about 10 cm² to about 25 cm².

In some embodiments of the methods disclosed herein, KX-01 is administered to an affected area of the subject that is about 16 cm² to about 625 cm², about 16 cm² to about 300 cm², is about 16 cm² to about 200 cm², about 16 cm² to about 100 cm², about 16 cm² to about 75 cm², about 16 cm² to about 50 cm², or about 16 cm² to about 25 cm² In some embodiments of the methods disclosed herein, KX-01 is administered to an affected area of the subject that is about 1 cm² to about 300 cm², about 1 cm² to about 200 cm², about 1 cm² to about 100 cm², about 1 cm² to about 75 cm², about 1 cm² to about 50 cm², or about 1 cm² to about 25 cm².

In some embodiments of the methods disclosed herein, KX-01 is administered to an affected area of the subject that is about 10 cm² to about 300 cm², about 10 cm² to about 200 cm², about 10 cm² to about 100 cm², about 10 cm² to about 75 cm², about 10 cm² to about 50 cm², or about 10 cm² to about 25 cm².

In some embodiments of the methods disclosed herein, KX-01 is administered to an affected area of the subject that is about 25 cm² to about 300 cm², about 25 cm² to about 200 cm², about 25 cm² to about 100 cm², about 25 cm² to about 75 cm² or 25 to 50 cm².

In some embodiments of the methods disclosed herein, KX-01 is administered to an affected area of the subject that is the affected area is about 25 cm² to about 100 cm², about 25 cm² to about 90 cm², about 25 cm² to about 80 cm², or about 25 cm² to about 70 cm², about 25 cm² to about 60 cm², about 25 cm² to about 50 cm², about 25 cm² to about 40 cm², or about 25 cm² to about 30 cm².

In some embodiments of the methods disclosed herein, KX-01 is administered to an affected area of the subject that is about 0.01 cm², 0.1 cm², 1 cm², 2 cm², 3 cm², 4 cm², 5 cm², 6 cm², 7 cm², 8 cm², 9 cm², 10 cm², 15 cm², 20 cm², 25 cm², 30 cm², 35 cm², 40 cm², 45 cm², 50 cm², 55 cm², 60 cm², 65 cm², 70 cm², 75 cm², 80 cm², 85 cm², 90 cm², 95 cm², or 100 cm².

In some embodiments of the methods disclosed herein, KX-01 is administered to an affected area of the subject that is about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², or about 100 cm².

In some embodiments of the methods disclosed herein, KX-01 is administered to an affected area of the subject, wherein the affected area is the skin.

In some embodiments of the methods disclosed herein, KX-01 is administered to an affected area of the subject, wherein the affected area of the skin is located at one or more locations independently selected from the scalp, forehead, forearm, face, nose, ears, eye lids, lips, neck, arms, elbows, hands, trunk, legs, knees, and feet.

In some embodiments of the methods disclosed herein, the subject has more than one affected area.

In some embodiments of the methods disclosed herein, the affected area is contiguous. In some embodiments of the methods disclosed herein, the affected area is non-contiguous.

In some embodiments of the methods disclosed herein, the subject has more than one affected area located at one or more locations independently selected from the scalp, forehead, forearm, face, nose, ears, eye lids, lips, neck, arms, elbows, hands, trunk, legs, knees, and feet.

In some embodiments of the methods disclosed herein, KX-01 is administered once a week, once every three days, once every two days, once a day, twice a day, three times a day, or four times a day.

In some embodiments of the methods disclosed herein, KX-01 is administered once a day or twice a day.

In some embodiments of the methods disclosed herein, KX-01 is administered once a day.

In some embodiments of the methods disclosed herein, KX-01 is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days.

In some embodiments of the methods disclosed herein, KX-01 is administered for 1, 2, 3, 4, 5, 6, 7, 8, 9, 10, 11, 12, 13, 14, 15, 16, 17, 18, 19, 20, or 21 days.

In some embodiments of the methods disclosed herein, KX-01 is administered for 1, 2, 3, 4, 5, 6, 7, 8, 9, 10, 11, 12, 13, or 14 days.

In some embodiments of the methods disclosed herein, KX-01 is administered for 1, 2, 3, 4, 5, 6, or 7 days.

In some embodiments of the methods disclosed herein, KX-01 is administered for 1, 2, 3, 4, or 5 days.

In some embodiments of the methods disclosed herein, KX-01 is administered for 2, 3, 4, 5, 6, 7, 8, 9, 10, 11, 12, 13, 14, or 28 days.

In some embodiments of the methods disclosed herein KX-01 is administered for 4, 5, 6, 7, 8, 9, 10, 11, 12, 13, 14, or 28 days.

In some embodiments of the methods disclosed herein, KX-01 is administered for 6, 7, 8, 9, 10, 11, 12, 13, 14, or 28 days.

In some embodiments of the methods disclosed herein, KX-01 is administered for 8, 9, 10, 11, 12, 13, 14, or 28 days.

In some embodiments of the methods disclosed herein, KX-01 is administered for 10, 11, 12, 13, 14, or 28 days.

In some embodiments of the methods disclosed herein, KX-01 is administered for 1 day.

In some embodiments of the methods disclosed herein, KX-01 is administered for 2 days.

In some embodiments of the methods disclosed herein, KX-01 is administered for 3 days.

In some embodiments of the methods disclosed herein, KX-01 is administered for 4 days.

In some embodiments of the methods disclosed herein, KX-01 is administered for 5 days.

In some embodiments of the methods disclosed herein, KX-01 is administered for 6 days.

In some embodiments of the methods disclosed herein, KX-01 is administered for 7 days.

In some embodiments of the methods disclosed herein, KX-01 is administered for 8 days.

In some embodiments of the methods disclosed herein, KX-01 is administered for 9 days.

In some embodiments of the methods disclosed herein, KX-01 is administered for 10 days.

In some embodiments of the methods disclosed herein, KX-01 is administered for 11 days.

In some embodiments of the methods disclosed herein, KX-01 is administered for 12 days.

In some embodiments of the methods disclosed herein, KX-01 is administered for 14 days.

In some embodiments of the methods disclosed herein, KX-01 is administered for 28 days.

In some embodiments of the methods disclosed herein, KX-01 is administered for 1, 2, 3, 4, 5, 6, or 7 days per week.

In some embodiments of the methods disclosed herein, KX-01 is administered for 2, 3, 4, 5, 6, or 7 days per week.

In some embodiments of the methods disclosed herein, KX-01 is administered for 3, 4, 5, 6, or 7 days per week.

In some embodiments of the methods disclosed herein, KX-01 is administered for 4, 5, 6, or 7 days per week.

In some embodiments of the methods disclosed herein, KX-01 is administered for 5, 6, or 7 days per week.

In some embodiments of the methods disclosed herein, KX-01 is administered for 6 or 7 days per week.

In some embodiments of the methods disclosed herein, KX-01 is administered for 7 days per week.

In some embodiments of the methods disclosed herein, KX-01 is administered for 6 days per week.

In some embodiments of the methods disclosed herein, KX-01 is administered for 5 days per week.

In some embodiments of the methods disclosed herein, KX-01 is administered for 1, 2, 3, 4, 5, or 6 days per week.

In some embodiments of the methods disclosed herein, KX-01 is administered for 2, 3, 4, 5, or 6 days per week.

In some embodiments of the methods disclosed herein, KX-01 is administered once or twice daily continuously for more than one day per week, followed by discontinuation of the administration for the rest of the week.

In some embodiments of the methods disclosed herein, KX-01 is administered once or twice daily every other day.

In some embodiments of the methods disclosed herein, KX-01 is administered once or twice daily every three days, every four days, every five days, every six days, or every seven days.

In some embodiments of the methods disclosed herein, KX-01 is administered once or twice daily for two days in a row every three days, every four days, every five days, every six days, or every seven days.

In some embodiments of the methods disclosed herein, KX-01 is administered once or twice daily for three days in a row every four days, every five days, every six days, or every seven days.

In some embodiments of the methods disclosed herein, KX-01 is administered once or twice daily for four days in a row every five days, every six days, or every seven days.

In some embodiments of the methods disclosed herein, KX-01 is administered once daily for seven days.

In some embodiments of the methods disclosed herein, KX-01 is administered once daily for fourteen days in a row.

In some embodiments of the methods disclosed herein, KX-01 is administered once daily for twenty-one days in a row.

In some embodiments of the methods disclosed herein, KX-01 is administered once daily for twenty-eight days in a row.

In some embodiments of the methods disclosed herein, KX-01 is administered once daily for fourteen days in a row followed by seven days of no administration.

In some embodiments of the methods disclosed herein, KX-01 is administered once daily for fourteen days in a row followed by seven days of no administration, wherein the seven days of no administration is followed by seven days of once daily administration of KX-01.

In some embodiments of the methods disclosed herein, KX-01 is administered once daily for fourteen days in a row followed by seven days of no administration, wherein the seven days of no administration is followed by fourteen days of once daily administration of KX-01. In some embodiments of the methods disclosed herein, KX-01 is administered once daily for fourteen days in a row followed by seven days of no administration, wherein the seven days of no administration is followed by seven days of twice daily administration of KX-01.

In some embodiments of the methods disclosed herein, KX-01 is administered once daily for fourteen days in a row followed by seven days of no administration, wherein the seven days of no administration is followed by fourteen days of twice daily administration of KX-01.

In some embodiments of the methods disclosed herein, KX-01 is administered twice daily for seven days.

In some embodiments of the methods disclosed herein, KX-01 is administered twice daily for fourteen days in a row.

In some embodiments of the methods disclosed herein, KX-01 is administered twice daily for twenty-one days in a row.

In some embodiments of the methods disclosed herein, KX-01 is administered twice daily for twenty-eight days in a row.

In some embodiments of the methods disclosed herein, KX-01 is administered twice daily for fourteen days in a row followed by seven days of no administration.

In some embodiments of the methods disclosed herein, KX-01 is administered twice daily for fourteen days in a row followed by seven days of no administration, wherein the seven days of no administration is followed by seven days of twice daily administration of KX-01.

In some embodiments of the methods disclosed herein, KX-01 is administered twice daily for fourteen days in a row followed by seven days of no administration, wherein the seven days of no administration is followed by fourteen days of twice daily administration of KX-01.

In some embodiments of the methods disclosed herein, KX-01 is administered twice daily for fourteen days in a row followed by seven days of no administration, wherein the seven days of no administration is followed by seven days of once daily administration of KX-01.

In some embodiments of the methods disclosed herein, KX-01 is administered twice daily for fourteen days in a row followed by seven days of no administration, wherein the seven days of no administration is followed by fourteen days of once daily administration of KX-01.

In some embodiments of the methods disclosed herein, KX-01 is administered until the psoriasis is fully treated.

In some embodiments of the methods disclosed herein, KX-01 is administered until the psoriasis is fully treated, i.e., the psoriasis is clear from the affected area of the subject.

In some embodiments of the methods disclosed herein, KX-01 is administered topically.

In some embodiments of the methods disclosed herein, KX-01 is administered at a concertation of about 0.1 mg/g once a day.

In some embodiments of the methods disclosed herein, KX-01 is administered at a concertation of about 0.1 mg/g once a day for 12 days.

In some embodiments of the methods disclosed herein, KX-01 is administered at a concertation of about 0.1 mg/g once a day for 14 days.

In some embodiments of the methods disclosed herein, KX-01 is administered at a concertation of about 0.1 mg/g once a day for 28 days.

In some embodiments of the methods disclosed herein, KX-01 is administered at a concertation of about 0.1 mg/g once a day for 5 days.

In some embodiments of the methods disclosed herein, KX-01 is administered at a concertation of about 0.1 mg/g twice a day.

In some embodiments of the methods disclosed herein, KX-01 is administered at a concertation of about 0.1 mg/g twice a day for 12 days.

In some embodiments of the methods disclosed herein, KX-01 is administered at a concertation of about 0.1 mg/g twice a day for 14 days.

In some embodiments of the methods disclosed herein, KX-01 is administered at a concertation of about 0.1 mg/g twice a day for 28 days.

In some embodiments of the methods disclosed herein, KX-01 is administered at a concertation of about 0.1 mg/g twice a day for 5 days.

In some embodiments of the methods disclosed herein, KX-01 is administered at a concertation of about 1.0 mg/g once a day.

In some embodiments of the methods disclosed herein, KX-01 is administered at a concertation of about 1.0 mg/g once a day for 12 days.

In some embodiments of the methods disclosed herein, KX-01 is administered at a concertation of about 1.0 mg/g once a day for 14 days.

In some embodiments of the methods disclosed herein, KX-01 is administered at a concertation of about 1.0 mg/g once a day for 28 days.

In some embodiments of the methods disclosed herein, KX-01 is administered at a concertation of about 1.0 mg/g once a day for 5 days.

In some embodiments of the methods disclosed herein, KX-01 is administered at a concertation of about 1.0 mg/g twice a day.

In some embodiments of the methods disclosed herein, KX-01 is administered at a concertation of about 1.0 mg/g twice a day for 12 days.

In some embodiments of the methods disclosed herein, KX-01 is administered at a concertation of about 1.0 mg/g twice a day for 14 days.

In some embodiments of the methods disclosed herein, KX-01 is administered at a concertation of about 1.0 mg/g twice a day for 28 days.

In some embodiments of the methods disclosed herein, KX-01 is administered at a concertation of about 1.0 mg/g twice a day for 5 days.

In some embodiments of the methods disclosed herein, KX-01 is administered at a concertation of about 10 mg/g once a day.

In some embodiments of the methods disclosed herein, KX-01 is administered at a concertation of about 10 mg/g once a day for 12 days.

In some embodiments of the methods disclosed herein, KX-01 is administered at a concertation of about 10 mg/g once a day for 14 days.

In some embodiments of the methods disclosed herein, KX-01 is administered at a concertation of about 10 mg/g once a day for 5 days.

In some embodiments of the methods disclosed herein, KX-01 is administered at a concertation of about 10 mg/g twice a day.

In some embodiments of the methods disclosed herein, KX-01 is administered at a concertation of about 10 mg/g twice a day for 12 days.

In some embodiments of the methods disclosed herein, KX-01 is administered at a concertation of about 10 mg/g once a day for 14 days.

In some embodiments of the methods disclosed herein, KX-01 is administered at a concertation of about 10 mg/g twice a day for 5 days.

In some embodiments of the methods disclosed herein, KX-01 is administered as described in the preceding paragraphs, followed by a first period during which KX-01 is not administered, further followed by a second period during which KX-01 is administered.

In some embodiments, the first period is 1 week, 2 weeks, 3 weeks, or 4 weeks. In some embodiments, the first period is 2 weeks. In some embodiments, the second period is 1 week, 2 weeks, 3 weeks, or 4 weeks. In some embodiments, the second period is 2 weeks. In some embodiments, KX-01 is administered during the second period at the same dose as KX-01 is administered before the first period. In some embodiments, KX-01 is administered during the second period at a different dose as KX-01 is administered before the first period.

In some embodiments of the methods disclosed herein, the administration of KX-01 reduces the number and/or severity of local skin reactions or other adverse side effects in the subject compared to other treatments of psoriasis, such as one described herein.

In some embodiments of the methods disclosed herein, the administration of KX-01 reduces the number of the subjects that have local skin reactions or other adverse side effects compared to other treatments of psoriasis, such as one described herein.

In some embodiments of the methods disclosed herein, the local skin reaction is selected from the group selected from vesiculation, postulation, erosion, ulceration, redness, swelling, flaking, scaling, hard lumps, dryness, pus, and blistering.

In some embodiments of the methods disclosed herein, the other side effect is selected from the group consisting of application site pain, application site pruritus, application site irritation, application site swelling, application site burning sensation, application site infection, periorbital edema, nasopharyngitis, chills, sore throat, drooping eyes, puffy eyes, hypopigmentation, hyperpigmentation, and headache.

In some embodiments, the disclosure pertains to KX-01 for use (e.g., topical use) in the treatment and/or prevention of psoriasis. In some embodiments, KX-01 is for use at the doses, dosing schedules, and/or one or more affected area in a subject in need thereof as described herein. In some embodiments, KX-01 is for use at the doses, dosing schedules, and one or more affected area in a subject in need thereof as described herein. In some embodiments, KX-01 is for use at the doses, dosing schedules, or one or more affected area in a subject in need thereof as described herein.

In some embodiments, KX-01 is for use at the doses as described herein. In some embodiments, KX-01 is for use at the dosing schedules as described herein. In some embodiments, KX-01 is for use at one or more affected area in a subject in need thereof as described herein.

In some embodiments, the disclosure pertains to use (e.g., topical use) of KX-01 in the treatment and/or prevention of psoriasis. In some embodiments, KX-01 is used at the doses, dosing schedules, and/or one or more affected area in a subject in need thereof as described herein. In some embodiments, KX-01 is used at the doses, dosing schedules, and one or more affected area in a subject in need thereof as described herein. In some embodiments, KX-01 is used at the doses, dosing schedules, or one or more affected area in a subject in need thereof as described herein.

In some embodiments, KX-01 is used at the doses as described herein. In some embodiments, KX-01 is used at the dosing schedules as described herein. In some embodiments, KX-01 is used at the one or more affected area in a subject in need thereof as described herein.

In some embodiments, the disclosure pertains to use of KX-01 in the manufacture of a medicament for the treatment and/or prevention of psoriasis. In some embodiments, KX-01 is used at the doses, dosing schedules, and/or one or more affected area in a subject in need thereof as described herein. In some embodiments, KX-01 is used at the doses, dosing schedules, and one or more affected area in a subject in need thereof as described herein. In some embodiments, KX-01 is used at the doses, dosing schedules, or one or more affected area in a subject in need thereof as described herein.

In some embodiments, KX-01 is used at the doses as described herein. In some embodiments, KX-01 is used at the dosing schedules as described herein. In some embodiments, KX-01 is used at the one or more affected area in a subject in need thereof as described herein.

Unless explicitly indicated otherwise, the terms “KX-01” and “KX2-391” refer to the basic form of the compound, i.e., the “free base,” which has the following structure:

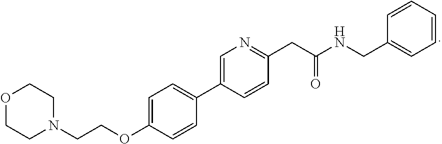 [SMILES: O=C(Cc1ccc(-c2ccc(OCCN3CCOCC3)cc2)cn1)NCc1ccccc1]

The term “KX-01 MSA” refers to the mesylate salt of KX-01, i.e., the salt compound resulting from reacting KX-01 with methane sulfonic acid.

“KX-01”, as used herein, may also be called “KX01”, “KX2-391”, or “KX-2-391”.

KX-01, and salts thereof, e.g., KX-01 MSA, and their preparation are disclosed in PCT Application Publication Nos. WO 2008/082637, WO 2008/144045, and WO 2010/135429. These publications are incorporated by reference herein in their entireties.

Psoriasis is a chronic autoimmune skin disease that speeds up the growth cycle of skin cells. Psoriasis causes localized or generalized patches of red papules and plaques, covered with white or silver scales and itching on the scalp, face, elbows, knees, and lower back. Psoriasis may also appear or other body parts, e.g., the hands, the feet, and other areas on the trunk and legs.

As used herein, the term “trunk” refers to the portion of a subject that is not an arm, a leg, or the head.

Psoriasis is most common in early adulthood subjects, e.g. about 15 years old to about 35 years old. Psoriasis may occur in children under the age of 10. A subject suffering from psoriasis may have a single lesion or multiple lesions. In some embodiments of the methods disclosed herein, KX-01 is administered to a subject with psoriasis between the ages of about 0 years old to about 110 years old. In some embodiments, the subject is between the ages of about 0 years old to about 10 years old. In some embodiments, the subject is between the ages of about 10 years old to about 20 years old. In some embodiments, the subject is between the ages of about 20 years old to about 30 years old. In some embodiments, the subject is between the ages of about 30 years old to about 40 years old. In some embodiments, the subject is between the ages of about 40 years old to about 50 years old. In some embodiments, the subject is between the ages of about 50 years old to about 60 years old. In some embodiments, the subject is between the ages of about 60 years old to about 70 years old. In some embodiments, the subject is between the ages of about 70 years old to about 80 years old. In some embodiments, the subject is between the ages of about 80 years old to about 90 years old. In some embodiments, the subject is between the ages of about 90 years old to about 100 years old. In some embodiments, the subject is between the ages of about 90 years old to about 110 years old. In some embodiments, the subject is between the ages of about 10 years old to about 40 years old. In some embodiments, the subject is between the ages of about 15 years old to about 40 years old. In some embodiments, the subject is between the ages of about 15 years old to about 35 years old. In some embodiments, the subject is between the ages of about 15 years old to about 30 years old. In some embodiments, the subject is between the ages of about 15 years old to about 25 years old. In some embodiments, the subject is between the ages of about 60 years old to about 110 years old. In some embodiments, the subject is between the ages of about 60 years old to about 100 years old. In some embodiments, the subject is between the ages of about 60 years old to about 90 years old. In some embodiments, the subject is between the ages of about 60 years old to about 80 years old. In some embodiments, the subject is between the ages of about 60 years old to about 70 years old.

In some embodiments, psoriasis can increase the risk of getting certain cancers. In some embodiments, the cancer is squamous cell carcinoma. In some embodiments, the cancer is lymphoma.

The five types of psoriasis include: plaque psoriasis (most common), guttate, inverse, pustular, and erythrodermic. Unless explicitly indicated otherwise, the methods described herein are applicable to all clinical variants, including those listed herein. Psoriasis has been associated with other serious health conditions, including but not limited to diabetes, heart disease, and depression.

In some embodiments, the psoriasis is plaque psoriasis, guttate, inverse, pustular, or erythrodermic. In some embodiments, the psoriasis is plaque psoriasis. In some embodiments, the psoriasis is guttate. In some embodiments, the psoriasis is inverse. In some embodiments, the psoriasis is pustular. In some embodiments, the psoriasis is erythrodermic.

Treatments for psoriasis include, but are not limited to, both topical treatments such as steroid creams, occlusion, light therapy, oral medications, and injectable medications. Topical treatments include topical corticosteroids, vitamin D analogues (Dovonex, Vectical), anthralin (Dritho-Scalp), topical retinoids (Tazorac, Avage), calcineurin inhibitors (Prograf and Elidel), salicyclic acid, coal tar, and moisturizers. Light therapy (phototherapy) treatments include: exposure to ultraviolet rays in sunlight or artificial light, UVB phototherapy, narrow band UVB phototherapy, Goeckerman therapy, Psoralen plus ultraviolet A (PUVA), and excimer laser. Treatments for psoriasis may also include a kinase inhibitor, an anti-immune response agent, or an anti-inflammatory agent (e.g., an inhibitor of phosphodiesterase 4 or an inhibitor of TNFα). Examples of the psoriasis treatments include Apremilast, Methotrexate, Tofacitinib, Alefacept, etanercept, Certolizumab-pegol, Guselkumab, Tildrakizumab, Risankizumab, Secukinumab, Ixekizumab, Brodalumab, Efalizumab, Adalimumab, Ustekinumab, and Infliximab. Oral or injectable treatments include: retinoids, methotrexate (rheumatrex), cyclosporine (Gengraf, Neoral), and biologics, such as etanercept (Enbrel), infliximab (Remicade), adalimumab (Humira), ustekinumab (Stelara), golimumab (Simponi), apremilast (Otezla), secukinumab (Cosentyx) and ixekizumab (Taltz). Other treatments include thioguanine (Tabloid) and hydroxyurea (Droxia, Hydrea). Alternative treatments include: aloe vera, fish oil, and Oregon grape.

The phrase “until the psoriasis clears,” as used herein, refers to the instance where the lesions on a subject suffering from psoriasis have substantially or completely disappeared from the treated area on the subject. In some embodiments, “substantially,” in this context, refers more than 50% of the psoriasis lesions have disappeared from the treated area on the subject. In some embodiments, “substantially” refers more than 60% of the psoriasis lesions have disappeared from the treated area on the subject. In some embodiments, “substantially” refers more than 70% of the psoriasis lesions have disappeared from the treated area on the subject. In some embodiments, “substantially” refers more than 80% of the psoriasis lesions have disappeared from the treated area on the subject. In some embodiments, “substantially” refers more than 90% of the psoriasis lesions have disappeared from the treated area on the subject. In some embodiments, “substantially” refers more than 95% of the psoriasis lesions have disappeared from the treated area on the subject. In some embodiments, “substantially” refers more than 99% of the psoriasis lesions have disappeared from the treated area on the subject.

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “comprising” is intended to mean that the method includes the recited elements, but do not exclude others. “Consisting essentially of” when used to define methods, shall mean excluding other elements of any essential significance to the combination when used for the intended purpose. Thus, a method consisting essentially of the elements as defined herein would not exclude substantial method steps. “Consisting of” shall mean excluding more than substantial method steps. Embodiments defined by each of these transition terms are within the scope of this disclosure.

Unless explicitly indicated otherwise, the terms “approximately” and “about” are synonymous. In some embodiments, “approximately” and “about” refer to the recited amount, value, or duration ±5%, ±4.5%, ±4%, ±3.5%, ±3%, ±2.5%, ±2%, ±1.75%, ±1.5%, ±1.25%, ±1%, ±0.9%, ±0.8%, ±0.7%, ±0.6%, ±0.5% ±0.4%, ±0.3%, ±0.2%, ±0.1%, ±0.09%, ±0.08%, ±0.07%, ±0.06%, ±0.05%, ±0.04%, ±0.03%, ±0.02%, or ±0.01%. In some embodiments, “approximately” and “about” refer to the listed amount, value, or duration ±2.5%, ±2%, ±1.75%, ±1.5%, ±1.25%, ±1%, ±0.9%, ±0.8%, ±0.7%, ±0.6%, ±0.5%. In some embodiments, “approximately” and “about” refer to the listed amount, value, or duration ±1%. In some embodiments, “approximately” and “about” refer to the listed amount, value, or duration ±0.5%. In some embodiments, “approximately” and “about” refer to the listed amount, value, or duration ±0.1/%.

The term “subject” includes any living organism that has psoriasis, or is at a risk of developing psoriasis. In some embodiments, the term “subject” refers to a mammal that has psoriasis, or is at a risk of developing psoriasis. In some embodiments, the term subject refers to a human being that has psoriasis, or is at a risk of developing psoriasis. The term “patient” is meant to be synonymous and may be used interchangeably with “subject,” unless explicitly indicated otherwise.

The term “therapeutically effective amount”, as used herein, refers to an amount of a pharmaceutical agent, e.g., KX-01, to treat, ameliorate, or prevent an identified disease or condition, e.g., psoriasis, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration.

Therapeutically effective amounts for a given situation can be determined by routine experimentation that is within the skill and judgment of the clinician.

For any compound, the therapeutically effective amount can be estimated in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. The dosage may vary within this range depending upon the dosage form employed and sensitivity of the subject.

Dosage and administration are adjusted to provide sufficient levels of the active ingredient or to maintain the desired effect. Factors which may be taken into account include the severity of the disease state, location of the disease on the subject, general health of the subject, age, weight, and gender of the subject, diet, time and frequency of administration, drug combination(s), reaction sensitivities, and tolerance/response to therapy. KX-01 may be administered every day, every other day, every three days, every four days, every five days, every six days, every week, biweekly, or once every two weeks depending on half-life and clearance rate.

For any of the methods described herein, KX-01 may be administered topically, intradermally, interepidermally, intragingivally, intraocularly, nasally, ophthalmically, percutaneously, periodontally, subconjuctivally, sublingually, transmucosally, or otically. In some embodiments, KX-01 may be administered topically.

All percentages and ratios used herein, unless otherwise indicated, are by weight.

Every document cited herein, including any cross referenced or related patent or application, is hereby incorporated herein by reference in its entirety unless expressly excluded or otherwise limited. The citation of any document is not an admission that it is prior art with respect to any subject matter disclosed or claimed herein or that it alone, or in any combination with any other reference or references, teaches, suggests or discloses any such subject matter.

Further, to the extent that any meaning or definition of a term in this document conflicts with any meaning or definition of the same term in a document incorporated by reference, the meaning or definition assigned to that term in this document shall govern.

While particular embodiments of the disclosure have been illustrated and described, various other changes and modifications can be made without departing from the spirit and scope of the disclosure. The scope of the appended claims includes all such changes and modifications that are within the scope of this disclosure.

Other features and advantages of the present disclosure are apparent from the different examples. The provided examples illustrate different components and methodology useful in practicing the present disclosure.

The examples do not limit the claimed disclosure. Based on the present disclosure the skilled artisan can identify and employ other components and methodology useful for practicing the present disclosure.

### Examples

### Example 1. Clinical Activity in Subjects with Psoriasis: Treatment with KX-01

Clinical activity signals were observed in the first cohort of subjects with psoriasis treated with KX-01 (Tirbanibulin) 1% ointment daily for five days in a phase I clinical trial.

Six subjects with psoriasis with Total Area Score (TAS) of 4-8 (based on the sum of the scores of erythema, elevation and scaling) were assessed. All subjects showed an improvement of their TAS score. One subject had complete resolution of skin scaling and another had improvement of psoriatic plaque thickness. The treatment was found to be well-tolerated. Only mild (grade-1) skin burning sensation and irritation were observed in one subject each. There was no significant absorption of KX-01 systemically. Good safety profile of KX-01 ointment in the treatment of psoriasis was observed in the study

Subjects in clinical studies of KX-01 ointment where administered KX-01 ointment up to 250 mg (5 mg/cm² [administration amount]×25 cm² [administration area]×2 [safety factor for larger application area]). This amount of the 0.01% or 0.1% concentration of ointment contains 0.025 mg or 0.25 mg of KX-01. Therefore, the amount of KX-01 delivered to the skin of the average 60 kg subject is 0.025 or 0.25 mg/60 kg/dose, or 0.00042 or 0.0042 mg/kg/dose. This dose is approximately 29700 or 2970 times lower than the 12.4 mg/kg/dose of KX-01 that caused mild to moderate skin irritation but no systemic adverse effects after six days of twice daily dermal dosing to rats. The safety margin in administered dermal KX-01 dose indicates little risk of adverse systemic effects in humans under the conditions of the clinical studies of KX-01 ointment based on nonclinical studies in animals.

### Example 2. Rat, Rabbit, and Mini-Pig KX01 Treatment

Twice daily administration of KX-01 ointment to rats (six days) and rabbits (seven days twice daily dosing with a single dose on day eight) at concentrations and amounts that delivered up to 12.4 (rats) or 2.0667 (rabbits) mg/kg KX-01 in ointment caused mild to moderate skin irritation, but no signs of systemic adverse effects. Plasma concentrations of KX-01 were very low after a single dose of KX-01 ointment, but after six days repeat dermal dosing, plasma concentrations reached or exceeded those seen at the No-Adverse-Effect dose in a 28-day oral toxicity study in rats.

KX-01 1% once daily administration of KX-01 ointment was examined on the mini-pigs on 10% of body surface area with skin being occluded for consecutive 28 days. The results showed very slight to slight edema, very slight to severe erythema and eschar, and/or desquamation, fissuring, scab, ulceration in the skin at the drug application site, decreased body weight, changes in hepatic, renal, and haematological profiles. Most of these findings were resolved by the last week of recovery.

### Example 3. Phase I Dose Escalation Study

A phase I dose escalation study is conducted to assess the safety, tolerability and activity of three different strengths of topical KX-01 in the treatment of subjects with plaque-type psoriasis. In stage I, each subject will be assigned randomly to KX-01 (0.01%, n=6) or placebo (n=2) before the first administration of investigational medicinal product (KX-01 or placebo). 22 subjects are recruited in three stages.

Each subject in stage I will receive treatment for two weeks, followed by one-week wash-out, another two-week treatment, and then two-week follow-up. After a satisfactory collaborative review for the safety data of the lower strength (0.01%) at the end of follow up for each subject, if there is no major safety concern (major safety concern is defined as ≥2 subjects in the KX-01 group having ≥CTCAE Grade 3 or severe adverse drug reaction), as well as an unanimous consent by the sponsor and the principle investigator(s), stage II study begins where each subject is assigned randomly to KX-01 (0.1%, n=6) or placebo (n=2) before the first administration.

Each subject in the stage II will receive treatment for four weeks, followed by a two-week follow-up. After a satisfactory collaborative review for the safety data of the lower strength (0.1%) at the end of follow up for each subject, if there is no major safety concern, stage III study follows. Stage III comprises a single arm study (n=6), where each subject will receive 1% KX-01 once daily for consecutive five days and then receive post-treatment follow-up on day 6, 15, and 29.
Stage I: 6 subjects (KX-01 0.01% [0.1 mg/g])+2 subjects (placebo)
Stage II: 6 subjects (KX-01 0.1% [1.0 mg/g])+2 subjects (placebo)
Stage III: 6 subjects (KX-01 1% [10 mg/g]) Subjects visit the study center for ambulatory visits:
Stage I (See Tables 1A and 1B)
   Visit 1: Screening, within 28 days before first application of IMP
   Visit 2: Week 1 (Day 1/Baseline)
   Visit 3: Week 2 (Day 8±3/After consecutive 1-week treatment)
   Visit 4: Week 3 (Day 15±3/After consecutive 2-week treatment)
   Visit 5: Week 4 (Day 22±3/After 1 week wash-out)
   Visit 6: Week 6 (Day 36±3/After another consecutive 2-week treatment)
   Visit 7: Week 8 (Day 50±3/After 2-week post-treatment follow-up)
Stage II (See Tables 2A and 2B)
   Visit 1: Screening, within 28 days before first application of IMP
   Visit 2: Week 1 (Day 1/Baseline)
Telephone interview (Day 8±3, after consecutive 1-week treatment)
   Visit 3: Week 3 (Day 15±3/After consecutive 2-week treatment)
   Visit 4: Week 5 (Day 29±3/After consecutive 4-week treatment)
   Visit 5: Week 7 (Day 43±3/After 2-week post-treatment follow-up)
Stage M (See Table 3)
   Visit 1: Screening, within 28 days before first application of IMP
   Visit 2: Day 1, baseline
   Visit 3: Day 6±1, after consecutive 5-day treatment
   Visit 4: Day 15±2, after 10-day post-treatment follow up
   Visit 5: Day 29±2, after 24-day post-treatment follow-up

The IMP (treatment or placebo) is applied to a single lesion. For stage I and II, subjects apply the IMP topically twice daily (at least four hours apart, suggested approximately 8-12 hours apart) to the selected treatment lesion regardless of improvement in the lesion. For stage I, each subject receives IMP for 14 consecutive days, followed by 1-week wash-out period and then receives IMP for another 14 consecutive days. For stage II, each subject receives IMP for 28 consecutive days. For stage III, each subject receives IMP once daily for consecutive five days. For stage I and II, blood samples for the determination of KX-01 concentrations are collected prior to the morning dose at the designated ambulatory visits to the study center (Stage I: Day 1, 8±3, 15±3, 22±3 Stage II: Day 1, 15±3, 29±3) (See Table 4). For stage III, blood samples for drug concentrations are collected prior to the dose on day 1, and day 6±1, day 15±2. The duration of this study is approximately three months for each subjects (See Table 4).

### Inclusion Criteria

Subjects who meet the following inclusion criteria are considered eligible to participate in the study:

1. Male and female subjects with plaque-type psoriasis, 20 years and older.

2. Subject had a confirmed diagnosis of chronic plaque-type psoriasis (without recent documented flare within 30 days prior to screening) for at least six months.

3. A single lesion of at least 16 cm² and no more than 625 cm² (≥16 cm² & ≤625 cm²) in size for stage I and II, and at least 16 cm² and no more than 100 cm² (≥16 cm² & ≤100 cm²) in size for stage III is selected as the target lesion (assessed at screening and Day 1). The lesion should be in an area sufficient for the application of the IMP and meet the following criteria:
Located on the trunk and/or the extremities, e.g. lesion located on the head, palms or soles of the feet. Intertriginous or genitoanal areas are not suitable.
No evidence of atrophy in the area selected for treatment.

4. Medical history, vital signs, physical examination, standard 12-lead electrocardiogram (ECG) and laboratory investigations must be clinically insignificant or within laboratory reference ranges for the relevant laboratory tests, unless the investigator considers the deviation for out of range values to be irrelevant for the purpose of the study.

5. No other disorders that, in the investigator's opinion, could prevent the subject from safely participating in this study or interfere with the evaluation of the subject's psoriasis.

6. Subject is able to discontinue the use of any systemic medication or therapy (e.g. oral or injectable psoriasis medications, psoralen plus long-wave ultraviolet [PUVA] therapy, herbal remedies, etc.) for psoriasis.

7. For females, either of the following conditions are to be met:
Not of childbearing potential:
   Surgically sterilized, undergone a hysterectomy, amenorrhea for
   ≥12 months and considered post-menopausal. Post-menopausal status is confirmed by evaluation of follicle stimulating hormone (FSH) (FSH >40 mIU/mL and estradiol <40 pg/mL [<147 pmol/L] are confirmatory).
Of childbearing potential:
   Negative serum pregnancy test at screening and not lactating. If this test is positive, the subject is excluded from the study. In the rare circumstance that a pregnancy is discovered after the subject received IMP, every attempt must be made to follow her to term.
   Either abstaining from sexual activity (if this is the usual lifestyle of the subject) or must agree to use an accepted method of contraception, and agree to continue with the same method throughout the study.
   Examples of reliable methods of contraception include oral contraceptive pill (documented that the dose has been stable for at least 4 weeks before the first intake of IMP), injectable or implantable contraceptives, intrauterine device, and barrier methods combined with an additional contraceptive method.
   Other methods, if considered by the investigator as reliable, is accepted.

8. Male subjects with partners of childbearing potential must be willing to use contraception during the study and three months after end of treatment and must not donate sperm for the duration of the study and for 3 months thereafter.

9. Subject must be able to provide written informed consent prior to the initiation of any study related procedures and able to comply with all the requirements of the study, including study visits and restrictions.

### Exclusion Criteria

Subjects who meet one or more of the exclusion criteria is not considered eligible to participate in the study.

1. History of hypersensitivity to the IMP or to medicinal products with similar chemical structures.

2. Presence of a skin disorder other than psoriasis in the target areas to be evaluated, including forms of inflammatory or non-inflammatory skin disorders that might interfere with determining efficacy or tolerability of the IMP.

3. Severe forms of psoriasis or forms of psoriasis other than plaque psoriasis.

4. All systemic psoriasis medications, including PUVA radiation treatments or other systemic immunosuppressive medication, are not allowed within five half-lives or 4 weeks (whichever is longer) prior to the first administration of the IMP, i.e. methotrexate, cyclosporine, PUVA, and corticosteroid (topical and oral) within 4 weeks prior to the first administration of the IMP.

5. The use of topical therapies for psoriasis, including ultraviolet light B, on the target lesion to be studied within two weeks prior to the first administration of the IMP. The use of topical calcipotriol at a dose up to 30 g per week or ultraviolet light B for psoriasis, or other non-systemically absorbed topical agents on non-treatment lesions is permitted during the study.

6. Previous treatment with anti-TNF/IL-12/IL-23 or any other monoclonal antibodies within three months prior to the first administration of the IMP.

7. Presence or history of any clinically significant acute or chronic disease which could interfere with the subject's participation or study outcome and at discretion of the clinical investigator.

8. Subject with drug-induced psoriasis and is unable to discontinue the causal agent(s)

9. Subject using prescription or non-prescription systemic drugs (e.g. vitamins and dietary, herbal supplements, Paracetamol, aspirin or non-steroidal anti-inflammatory drugs (NSAIDs)) that might have an effect on psoriasis and is unable to maintain the stable dose or discontinue the dose during the study period.

10. Participation in another study with an experimental drug, where the last administration of the previous IMP was within 4 weeks (or within five elimination half-lives for chemical entities or two elimination half-lives for antibodies or insulin, whichever is longer) before administration of IMP in this study, at the discretion of the investigator.

11. A positive serum pregnancy test (beta human chorionic gonadotropin [β-HCG]) or lactation.

12. Vulnerable subjects, e.g., persons in detention.

### Treatment Period

On day 1, a single treatment lesion consisting of active psoriasis skin lesion of at least 16 cm² in size with upper limit of 625 cm² for stage I and II, and at least 16 cm² and no more than 100 cm² (≥16 cm² &≤100 cm²) in size for stage III is selected for each subject. Skin area for IMP application is marked and checked at every visit.

For stage I and II, subjects receive twice daily strengths of 0.01% KX-01 (state I) or 0.1% KX-01 (stage II) or placebo during the treatment period. For stage III, subjects receive once daily strength of 1% KX-01 ointment for consecutive five days.

### Follow-Up

For stage I, and 11, subjects visit the study center two weeks after last dose of IMP to assess any new or ongoing AEs and skin lesions. For stage 111, subjects receive post-treatment follow-up on day 6, 15, and 29.

### Target Area Score

Change in Target Area Score (TAS) between baseline and EOT is measured for the target lesion. The TAS is performed by the investigator or a suitable trained designee, and whenever possible, the TAS for an individual subject is completed by the same assessor at all time-points.

At baseline and at specified time-points, the investigator evaluates the individual signs of the lesion including erythema, plaque elevation and scaling on a five-point scale: 0 (absent), 1 (mild), 2 (moderate), 3 (severe) and 4 (very severe). The TAS, which may range from 0 to 12, corresponds to the sum of the above three variables including erythema. plaque elevation and scaling.

TABLE 1A
Stage I Schedule of Assessment

| | | | | | | | | Early |
|---|---|---|---|---|---|---|---|---|
| | | | | | | | | Termination |
| | | | | | | | | Visit |
| | | | | | End of | | | Patient withdrew |
| | Screening | | | | Wash-out | Treatment | Follow- | consent or |
| | Period | Treatment Period 1 | | | Visit | Period 2 | up Visit | was withdrawn |
| | Within 28 | Day | Day | Day | Day | Day | Day | from the clinical |
| Evaluation | days | 1 | 8 ± 3 | 15 ± 3 | 22 ± 3 | 36 ± 3 | 50 ± 3 | study + 7 |
| Informed consent | X | | | | | | | |
| Inclusion/exclusion | X | X | | | | | | |
| criteria | | | | | | | | |
| Demographic | X | | | | | | | |
| information | | | | | | | | |
| Medical/ | X | X | | | | | | |
| medication/ | | | | | | | | |
| surgical history/ | | | | | | | | |
| dermatological | | | | | | | | |
| history | | | | | | | | |
| Define selected | X | X | | | | | | |
| treatment lesion | | | | | | | | |
| Physical | X | X | X | X | X | X | X | X |
| Examination | | | | | | | | |
| Vital Signs | X | X | X | X | X | X | X | X |
| 12-lead ECG | X | | | X | | X | | |
| Randomization | | X | | | | | | |
| Clinical laboratory | X | | | X | | X | | |
| tests (hematology, | | | | | | | | |
| chemistry, | | | | | | | | |
| urinalysis) | | | | | | | | |
| Pregnancy test | X (serum) | X (urine) | X (urine) | X (urine) | X (urine) | X (urine) | X (urine) | X (urine) |
| (females of | | | | | | | | |
| childbearing | | | | | | | | |
| potential only) | | | | | | | | |
| Target area score | X | X | X | X | X | X | X | X |
| (TAS) | | | | | | | | |
| Physician's Global | | X | X | X | X | X | X | X |
| Assessment of | | | | | | | | |
| target lesion | | | | | | | | |

ECG = electrocardiogram;
IMP = investigational medicinal product;
TAS = target area score

TABLE 1B
Stage I Schedule of Assessment

| | | | | | | | | Early |
|---|---|---|---|---|---|---|---|---|
| | | | | | | | | Termination |
| | | | | | | | | Visit |
| | | | | | End of | | | Patient withdrew |
| | Screening | Treatment Period | | | Wash-out | Treatment | Follow- | consent of |
| | Period | | (Telephone) | | visit | Period 2 | up Visit | was withdrawn |
| | Within 28 | Day | Day | Day | Day | Day | Day | from the clinical |
| Evaluation | days | 1 | 8 ± 3 | 15 ± 3 | 22 ± 3 | 36 ± 3 | 50 ± 3 | study + 7 |

| | | | | | | | |
|---|---|---|---|---|---|---|---|
| Local tolerability | X | X | X | X | X | X | X |
| score | | | | | | | |
| Standard color | X (pre | X (pre | X | X (pre | X | X | X |
| photography | first | morning | | morning | | | |
| dose) | dose) | | dose) | | | | |
| Blood sampling for | X (pre | X (pre | X | X (pre | X | | |
| KX-01 plasma | first | morning | | morning | | | |
| concentration | dose) | dose) | | dose) | | | |
| measurement | | | | | | | |
| Dispense IMP and | X | X | | X | | | |
| Diary card to patient | | | | | | | |
| IMP and Diary Card | | X | X | | X | | |
| retum/accountability | | | | | | | |
| assessment | | | | | | | |
| Telephone Interview | | X | | | | | |

| | | |
|---|---|---|
| IMP dosing | [-----------------X-----------------] | [----X----] |

| | | |
|---|---|---|
| Prior and | [------------------------X------------------------] | X |
| concomitant | | |
| medications | | |
| Adverse events | [------------------------X------------------------] | X |

TABLE 2A
Stage II Schedule of Assessments

| | | | | | | | Early |
|---|---|---|---|---|---|---|---|
| | | | | | | | Termination |
| | | | | | | | Visit |
| | | | | | | | Patient withdrew |
| | Screening | Treatment Period | | | | Follow- | consent or |
| | Period | | (Telephone) | | | up Visit | was withdrawn |
| | Within 28 | Day | Day | Day | Day | Day | from the clinical |
| Evaluation | days | 1 | 8 ± 3 | 15 ± 3 | 29 ± 3 | 43 ± 3 | study + 7 |

| | | | | | | |
|---|---|---|---|---|---|---|
| Informed consent | X | | | | | |
| Inclusion/exclusion | X | X | | | | |
| criteria | | | | | | |
| Demographic | X | | | | | |
| information | | | | | | |
| Medical/medication/ | X | X | | | | |
| surgical history/ | | | | | | |
| dermatological | | | | | | |
| history | | | | | | |
| Define selected | X | X | | | | |
| treatment lesion | | | | | | |
| Physical Examination | X | X | X | X | X | X |
| Vital Signs | X | X | X | X | X | X |
| 12-lead ECG | X | | X | X | | |
| Randomization | | X | | | | |
| Clinical laboratory | X | | X | X | | |
| tests (hematology, | | | | | | |
| chemistry, urinalysis) | | | | | | |
| Pregnancy test | X (serum) | X (urine) | X (urine) | X (urine) | X (urine) | X (urine) |
| (females of | | | | | | |
| childbearing potential | | | | | | |
| only) | | | | | | |
| Target area score | X | X | X | X | X | X |
| (TAS) | | | | | | |
| Physician's Global | | X | X | X | X | X |
| Assessment of target | | | | | | |
| lesion | | | | | | |
| Local Tolerability | | X | X | X | X | X |
| Score | | | | | | |
| Standard Color | | X (pre | X (pre | X | X | X |
| Photography | | first | morning | | | |
| | | dose) | dose) | | | |
| Disease relapse | | | | | X | |
| Blood sampling for | | X (pre | X (pre | X | | |
| KX-01 plasma | | first | morning | | | |
| concentration | | dose) | dose) | | | |
| measurement | | | | | | |

TABLE 2B
Stage II Schedule of Assessments

| | | | | | | | Early |
|---|---|---|---|---|---|---|---|
| | | | | | | | termination |
| | | | | | | | Patient withdrew |
| | Screening | Treatment Period | | | | Follow- | consent of |
| | Period | | (Telephone) | | | up Visit | was withdrawn |
| | Within 28 | Day | Day | Day | Day | Day | from the clinical |
| Evaluation | days | 1 | 8 ± 3 | 15 ± 3 | 29 ± 3 | 43 ± 3 | study + 7 |

| | | | | |
|---|---|---|---|---|
| Dispense IMP and | X | | X | |
| Diary card to patient | | | | |
| IMP and Diary Card | | | X | X |
| return/accountability | | | | |
| assessment | | | | |
| Telephone Interview | | X | | |

| | |
|---|---|
| IMP dosing | [--------------------X--------------------] |

| | | |
|---|---|---|
| Prior and | [------------------------X------------------------] | X |
| concomitant | | |
| medications | | |
| Adverse events | [------------------------X------------------------] | X |

ECG = electrocardiogram;
IMP = investigational medicinal product;
TAS = target area score

TABLE 3
Stage III Schedule of Assessment

| | VI | | | | | | |
|---|---|---|---|---|---|---|---|
| | (Screening) | | | | | | Early |
| | Within 28 | V2 | | V3 | V4 | V5 | Termination |
| Evaluation | days | Day 1 | Day 2-5 | Day 6 ± 1 | Day 15 ± 2 | Day 29 ± 2 | +2 |

| | | | | | | |
|---|---|---|---|---|---|---|
| Informed consent | X | | | | | |
| Inclusion/exclusion | X | X | | | | |
| criteria | | | | | | |
| Demographic | X | | | | | |
| information | | | | | | |
| Medical/ | X | X | | | | |
| medication/ | | | | | | |
| surgical history/ | | | | | | |
| dermatological | | | | | | |
| history | | | | | | |
| Define selected | X | X | | | | |
| treatment lesion | | | | | | |
| Physical | X | X | X | X | X | X |
| Examination | | | | | | |
| Vital Signs | X | X | X | X | X | X |
| 12-lead ECG | X | | X | X | X | X |
| Clinical laboratory | X | | X | X | X | X |
| tests (hematology, | | | | | | |
| chemistry, urinalysis) | | | | | | |
| Pregnancy test | X (serum) | X (urine) | X (urine) | X (urine) | X (urine) | X (urine) |
| (females of | | | | | | |
| childbearing | | | | | | |
| potential only) | | | | | | |
| Target area score | X | X | X | X | X | X |
| (TAS) | | | | | | |
| Physician's Global | | X | X | X | X | X |
| Assessment of target | | | | | | |
| lesion | | | | | | |
| Local tolerability | | X | X | X | X | X |
| score | | | | | | |
| Standard color | | X (pre | X | X | X | X |
| photography | | first | | | | |
| | | dose) | | | | |
| Disease relapse | | | | X | X | |
| Blood sampling for | | X (pre | X | X | | |
| KX-01 plasma | | first | | | | |
| concentration | | dose) | | | | |
| measurement | | | | | | |
| Dispense IMP and | | X | | | | |
| Diary card to patient | | | | | | |
| IMP and Diary Card | | | X | X | | |
| return/accountability | | | | | | |
| assessment | | | | | | |
| IMP dosing | | X | | | | |
| Prior and | X | X | X | X | X | X |
| concomitant | | | | | | |
| medications | | | | | | |
| Adverse events | X | X | X | X | X | X |

ECG = electrocardiogram;
IMP = investigational medicinal product;
TAS = target area score

1. The recorded medical history is updated if necessary on Day 1.
2. Including height and weight at screening.
3. Systolic and diastolic blood pressure, pulse and body temperature, and respiration rate are recorded.
4. Standard 12-lead ECG is performed.
5. Hematology (Ethylenediaminetetraacetic acid [EDTA tubes]): WBC, RBC, hemoglobin, hematocrit, platelets, neutrophils, eosinophils, basophil, monocyte and lymphocytes (differential, absolute and percentage), mean corpuscular volume (MCV), mean corpuscular haemoglobin (MCH), mean corpuscular haemoglobin concentration (MCHC). Clinical chemistry (Serum separator tubes [SST]): Potassium, sodium, urea, creatinine, uric acid, calcium, protein, albumin, total bilirubin, alkaline phosphatase (ALP), gamma glutamyl transferase (GGT), aspartate aminotransferase (AST), alanine aminotransferase (ALT) and AC glucose, triglycerides, bilirubin, cholesterol.
Urinalysis (dipstick): PH, protein, glucose, ketone, bilirubin, urobilinogen, blood, nitrite, leucocytes and specific gravity. If necessary, light microscopy—erythrocytes, leucocytes, hyaline casts, cellular casts, granular casts and epithelial cells.
6. Serum pregnancy test (quantitative-HCG [beta human chorionic gonadotropin] method) at screening. For all other visits, urine pregnancy testing is performed. Post-menopausal status is confirmed by evaluation of follicle stimulating hormone (FSH) (FSH >40 mIU/mL and estradiol <40 pg/mL [<147 pmol/L] are confirmatory).

### 7. See Section 7.2.10.

8. Drug level measurement blood samples of ˜10 mL are collected.
9. The telephone interview is conducted by the study coordinator on Day 8±3 to assess the drug compliance and any adverse drug reaction. If any severe (>Grade 2) adverse drug reaction is suspected, the earlier unscheduled visit is arranged if possible.
10. For stage III, the treatment duration is very short and any window allowance causes a significant difference in treatment duration. Thus, the subject is required to apply KXO1 dose for consecutive 5 days exactly. No window is allowed.
11. For the subject coming for the visit on Day 5, blood specimen for PK is drawn before the final dose.

TABLE 4
Blood Volume

| | Sample | Number of | |
|---|---|---|---|
| Assessment | Volume | Samples | Total Volume |
| Hematology | ~4 mL | 3 (Stage I & II) | ~12 mL |
| | | 4 (Stage III) | ~16 mL |
| Clinical | ~5 mL | 3 (Stage I & II) | ~15 mL |
| chemistry* | | 4 (Stage III) | ~20 mL |
| FSH | ~5 mL | 1 | ~5 mL |
| (postmenopausal | | | |
| females only) | | | |
| KX-01 plasma | ~10 mL | 4 (Stage I) | ~40 mL |
| concentration | | 3 (Stage II & III)) | ~30 mL |
| measurement | | | |
| Total | | | 57 m: (Stage II)~67 |
| | | | mL (Stage I): Post- |
| | | | menopausal: 62 mL |
| | | | (Stage II)~72 mL |
| | | | (Stage I) |
| | | | 66-71 mL for Stage |
| | | | III |

*Serum pregnancy test (females only) at screening will be performed on the sample collected for clinical chemistry

### Example 4. KXO1 Dosage Results

Most subjects using the KX-01 ointment 0.01% twice daily dose regimen demonstrated excellent and good tolerability. Only one treatment-emergent adverse event, Grade 1 tenderness, was related to drug. No serious adverse event (SAE) were reported.

### EQUIVALENTS

The present disclosure can be embodied in other specific forms without departing from the spirit or essential characteristics thereof. The foregoing embodiments are therefore to be considered in all respects illustrative rather than limiting on the present disclosure described herein. Scope of the present disclosure is thus indicated by the appended claims rather than by the foregoing description, and all changes that come within the meaning and range of equivalency of the claims are intended to be embraced therein.

1. A method of treating and/or preventing psoriasis comprising administering to a subject in need thereof a therapeutically effective amount of KX-01: 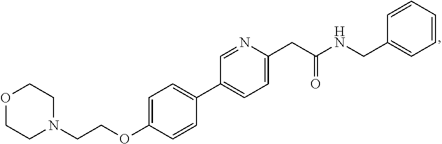 [SMILES: O=C(Cc1ccc(-c2ccc(OCCN3CCOCC3)cc2)cn1)NCc1ccccc1]
or a pharmaceutically acceptable salt thereof.

2. The method of claim 1, wherein KX-01 is administered to an affected area of the subject at a dose from about 0.1 mg to about 10 mg, from about 0.2 mg to about 5 mg, or from about 0.5 mg to about 2.5 mg.

3. The method of claim 1, wherein KX-01 is administered to an affected area of the subject at a dose of about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 4 mg, or about 5 mg.

4. The method of claim 1, wherein KX-01 is administered to an affected area of the subject at a dose from about 0.0003 mg/cm² to about 10 mg/cm², from about 0.001 mg/cm² to about 0.4 mg/cm², from about 0.005 mg/cm² to about 0.1 mg/cm², from about 0.005 mg/cm² to about 0.02 mg/cm², or from about 0.025 mg/cm² to about 0.1 mg/cm².

5. The method of claim 1, wherein KX-01 is administered to an affected area of the subject at a dose of about 0.001 mg/cm², about 0.002 mg/cm², about 0.003 mg/cm², about 0.004 mg/cm², about 0.005 mg/cm², about 0.006 mg/cm², about 0.007 mg/cm², about 0.008 mg/cm², about 0.009 mg/cm², about 0.01 mg/cm², about 0.02 mg/cm², about 0.03 mg/cm², about 0.04 mg/cm², about 0.05 mg/cm², about 0.06 mg/cm², about 0.07 mg/cm², about 0.08 mg/cm², about 0.09 mg/cm², about 0.1 mg/cm², about 0.15 mg/cm², about 0.2 mg/cm², about 0.25 mg/cm², about 0.3 mg/cm², about 0.35 mg/cm², or about 0.4 mg/cm².

6. The method of claim 2, wherein the affected area is about 0.01 cm² to about 300 cm², about 1 cm² to about 200 cm², about 1 cm² to about 100 cm², about 1 cm² to about 75 cm², about 1 cm² to about 50 cm², about 1 cm² to about 25 cm², about 10 cm² to about 200 cm², about 10 cm² to about 100 cm², about 10 cm² to about 75 cm², about 10 cm² to about 50 cm², about 10 cm² to about 25 cm², about 25 cm² to about 200 cm², about 25 cm² to about 100 cm², about 25 cm² to about 75 cm², or about 25 cm² to about 50 cm², about 25 cm² to about 90 cm², about 25 cm² to about 80 cm², or about 25 cm² to about 70 cm², about 25 cm² to about 60 cm², about 25 cm² to about 40 cm², or about 25 cm² to about 30 cm².

7. The method of claim 2, wherein the affected area is about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², or about 100 cm².

8. The method of claim 2, wherein the affected area is the skin.

9. The method of claim 1, wherein KX-01 is administered once a week, once every three days, once every two days, once a day, twice a day, three times a day, or four times a day.

10. The method of claim 1, wherein KX-01 is administered for 1, 2, 3, 4, 5, 6, 7, 8, 9, 10, 11, 12, 13, 14, 15, 16, 17, 18, 19, 20, or 21 days.

11. The method of claim 1, wherein KX-01 is administered for 1, 2, 3, 4, or 5 days.

12. The method of claim 1, wherein KX-01 is administered for 1, 2, 3, 4, 5, or 6 days per week.

13. The method of claim 1, wherein KX-01 is administered once or twice daily continuously for more than one day per week, followed by discontinuation of the administration for the rest of the week.

14. The method of claim 1, wherein KX-01 is administered once or twice daily every other day, every three days, every four days, every five days, every six days, or every seven days.

15. The method of claim 1, wherein KX-01 is administered once or twice daily for two days in a row every three days, every four days, every five days, every six days, or every seven days.

16. The method of claim 1, wherein KX-01 is administered once or twice daily for three days in a row every four days, every five days, every six days, or every seven days.

17. The method of claim 1, wherein KX-01 is administered once or twice daily for four days in a row every five days, every six days, or every seven days.

18. The method of claim 1, wherein KX-01 is administered until the psoriasis is fully treated.

19. The method of claim 1, wherein KX-01 is administered topically.

20. The method of claim 1, wherein the administration of KX-01 reduces the number and/or severity of, or the number of the subjects that have, local skin reactions or other adverse side effects in the subject compared to other treatments of psoriasis.

21. A compound for use in treating or preventing psoriasis comprising administering to a subject in need thereof a therapeutically effective amount of KX-01: 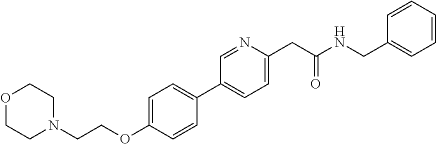 [SMILES: O=C(Cc1ccc(-c2ccc(OCCN3CCOCC3)cc2)cn1)NCc1ccccc1]
or a pharmaceutically acceptable salt thereof.

22. Use of a compound in the manufacture of a medicament for use in treating or preventing psoriasis comprising administering to a subject in need thereof a therapeutically effective amount of KX-01: 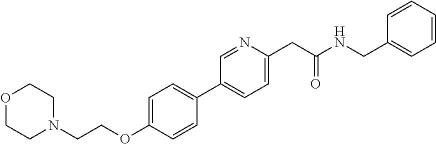 [SMILES: O=C(Cc1ccc(-c2ccc(OCCN3CCOCC3)cc2)cn1)NCc1ccccc1]
or a pharmaceutically acceptable salt thereof.

23. Use of a compound in treating or preventing psoriasis comprising administering to a subject in need thereof a therapeutically effective amount of KX-01: 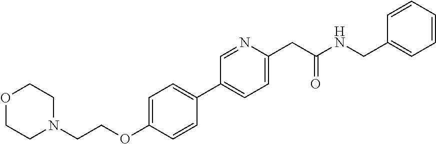 [SMILES: O=C(Cc1ccc(-c2ccc(OCCN3CCOCC3)cc2)cn1)NCc1ccccc1]
or a pharmaceutically acceptable salt thereof.
